(19) United States
(12) Patent Application Publication
(10) Pub. No.: US 2025/0099542 A1
(43) Pub. Date: Mar. 27, 2025

(54) METHOD OF TREATING A TUMOR WITH A COMBINATION OF IL-7 PROTEIN AND VEGF ANTAGONIST

(71) Applicants: NEOIMMUNETECH, INC., Rockville, MD (US); GENEXINE, INC., Seongnam-si, Gyeonggi-do (KR)

(72) Inventors: Donghoon CHOI, Seongnam-si (KR); Sun-Kyoung IM, Pohang-si (KR); Mankyu JI, Pohang-si (KR); Minji LEE, Pohang-si (KR); Seungtae BAEK, Pohang-si (KR); Jung Won WOO, Seoul (KR); Min Kyu HEO, Hwaseong-si (KR); Hee Won KIM

(73) Assignees: NEOIMMUNETECH, INC., Rockville, MD (US); GENEXINE, INC., Seongnam-si, Gyeonggi-do (KR)

(21) Appl. No.: 18/725,517

(22) PCT Filed: Dec. 30, 2022
(86) PCT No.: PCT/US2022/082639

§ 371 Date: Jun. 28, 2024

Related U.S. Application Data
(60) Provisional application No. 63/295,336, filed on Dec. 30, 2021.

(30) Foreign Application Priority Data
Jan. 3, 2022 (KR) 10-2022-0000051

(51) Int. Cl. A61K 38/20; A61K 39/00; A61K 39/395; A61P 35/00; C07K 16/22

(52) U.S. Cl. CPC A61K 38/2046; A61K 39/39533; A61P 35/00; C07K 16/22; A61K 2039/505; A61K 2039/545; C07K 2317/24

(57) ABSTRACT

The present disclosure relates to methods of treating a cancer (or a tumor) with an IL-7 protein in combination with a VEGF antagonist. In some aspects, the methods provided herein further comprise administering an additional therapeutic agent (e.g., immune checkpoint inhibitor) to the subject.

### CROSS-REFERENCE TO RELATED APPLICATION

This PCT application claims the priority benefit of U.S. Provisional Application No. 63/295,336, filed Dec. 30, 2021, and Korean Patent Application No. 10-2022-0000051, filed Jan. 3, 2022, which is herein incorporated by reference in its entirety.

### REFERENCE TO SEQUENCE LISTING SUBMITTED ELECTRONICALLY VIA EFS-WEB

The content of the electronically submitted sequence listing in .XML file (Name: 4241_029PC02_SequenceListing_ST26; Size: 122,317 bytes; and Date of Creation: Dec. 30, 2022) filed with the application is herein incorporated by reference in its entirety.

### FIELD OF DISCLOSURE

The present disclosure generally relates to combination treatment regimens that are useful for the treatment of tumors in a subject, e.g., human subject. Specifically, the treatment regimens provided herein comprise an IL-7 protein in combination with a VEGF antagonist. In some aspects, the treatment regimens further comprises additional therapeutic agents, such as immune checkpoint inhibitors. Provided herein are methods of treating a tumor, including increasing an anti-tumor immune response, using such treatment regimens.

### BACKGROUND OF THE DISCLOSURE

Human cancers harbor numerous genetic and epigenetic alterations, generating neoantigens potentially recognizable by the immune system. Sjoblom et al., Science 314:268-74 (2006). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Cancer immunotherapy has become well-established in recent years and is now one of the more successful treatment options available for many cancer patients. Scott, A. M., et al., Cancer Immun 12:14 (2012). Aside from targeting antigens that are involved in cancer cell proliferation and survival, antibodies can also activate or antagonize immunological pathways that are important in cancer immune surveillance. And, intensive efforts have led to the successful development of several immune checkpoint pathway inhibitors, some of which have been approved by the Food and Drug Administration, e.g., anti-CTLA-4 antibody: ipilimumab (YERVOY®); anti-PD-1 antibody: nivolumab (OPDIVO®), pembrolizumab (KEYTRUDA®); and anti-PD-L1 antibody: atezolizumab (TECENTRIQ®), durvalumab (IMFINZI®), avelumab (BAVENCIO®).

Despite such advances, patients with certain malignant tumors (e.g., metastatic or refractory solid tumors) continue to have very poor prognosis. Only a subset of such patients actually experience long-term cancer remission, with many patients either not responding or initially responding but eventually developing resistance to the antibodies. Sharma, P., et al., Cell 168(4): 707-723 (2017). Moreover, many cancer patients are lymphopenic, as many of the available standard of care cancer treatments (e.g., chemotherapy and radiation therapy) are known to cause lymphopenia. Grossman, S. A., et al., J Natl Compr Canc Netw 13(10):1225-31 (2015). Checkpoint inhibitors, such as anti-PD-1 antibodies, have been shown to have limited efficacy in such cancer patients. Yarchoan, M., et al., J Clin Oncol 35:e14512 (2017). Accordingly, there remains a need for new treatment options with acceptable safety profile and high efficacy in cancer patients, including those with lymphopenia.

### SUMMARY OF THE DISCLOSURE

Provided herein is a method of treating a tumor in a subject in need thereof, comprising administering to the subject an IL-7 protein in combination with an antagonist of a vascular endothelial growth factor (“VEGF”) (“VEGF antagonist”).

In some aspects, a tumor volume is reduced in the subject after the administration. In some aspects, the tumor volume is reduced by at least about 5%, at least about 10%, at least about 20%, at least about 30%, at least about 40%, at least about 50%, at least about 60%, at least about 70%, at least about 80%, at least about 90%, or about 100% after the administration.

Also provided herein is a method of increasing an anti-tumor immune response in a subject in need thereof, comprising administering to the subject an IL-7 protein in combination with an antagonist of a vascular endothelial growth factor (“VEGF”) (“VEGF antagonist”).

In some aspects, the anti-tumor immune response in the subject is increased by at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 40-fold, or at least about 50-fold after the administration. In some aspects, an increased anti-tumor immune response comprises: (i) a decrease in a tumor volume; (ii) an increase in an effector activity of a tumor-specific T cell; (iii) an increase in the number of tumor-specific effector T cells; (iv) an increase in the number of tumor-infiltrating lymphocytes (TILs) in a tumor; (v) a decrease in the number of myeloid-derived suppressor cells (MDSCs) in a tumor; (vi) a decrease in the number of regulatory T cells (Tregs) in a tumor; (vii) an increase in duration of survival of the subject; (viii) a decrease in expression of TOX on CD8+ T cells in a tumor, tumor-draining lymph node (TDLN), or both; (ix) an increase in the number of stem-like T cells in a tumor, TDLN, or both; or (x) a combination thereof.

In some aspects, the IL-7 protein and the VEGF antagonist are administered to the subject concurrently. In some aspects, the IL-7 protein and the VEGF antagonist are administered to the subject sequentially.

In some aspects, an additional therapeutic agent is administered to the subject. In some aspects, the additional therapeutic agent comprises an immune checkpoint inhibitor, an immune checkpoint activator, a standard care of treatment, or a combination thereof.

In some aspects, the immune checkpoint inhibitor comprises a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or a combination thereof. In some aspects, the immune checkpoint activator comprises an OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or a combination thereof. In some aspects, the standard care of treatment comprises a chemotherapy, radiation, or both.

In some aspects, the IL-7 protein is not a wild-type IL-7.

In some aspects, the IL-7 protein comprises an oligopeptide consisting of 1 to 10 amino acid residues. In some aspects, the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof. In some aspects, the oligopeptide is methionine-glycine-methionine (MGM).

In some aspects, the IL-7 protein comprises a half-life extending moiety. In some aspects, the half-life extending moiety comprises an Fc, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, or a combination thereof. In some aspects, wherein the half-life extending moiety is a Fc.

In some aspects, the Fc is a hybrid Fc, comprising a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to the sequence set forth in any one of SEQ ID NOs: 1-6, 15-26, and 80-85.

In some aspects, the VEGF antagonist comprises an antibody, or an antigen-binding fragment thereof, that specifically binds to the VEGF (“anti-VEGF antibody”), a polynucleotide encoding the anti-VEGF antibody, an antisense oligonucleotide, a siRNA, a shRNA, a miRNA, a dsRNA targeting VEGF, an aptamer, a PNA, or a vector comprising thereof. In some aspects, the VEGF antagonist is an anti-VEGF antibody. In some aspects, the anti-VEGF antibody comprises aflibercept (EYLEA® and ZALTRAP®), bevacizumab (AVASTIN®, ZIRABEV®, and MVASI®), ranibizumab (LUCENTIS®, BYOOVIZ®, SUSVIM®), or a combination thereof.

In some aspects, the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof. In some aspects, the brain cancer comprises a glioblastoma. In some aspects, the glioblastoma comprise a recurrent glioblastoma. In some aspects, the glioblastoma is refractory to a standard of care therapy. In some aspects, the skin cancer comprises a Merkel cell carcinoma (MCC), basal cell carcinoma (BCC), cutaneous squamous cell carcinoma (cSCC), melanoma, or a combination thereof.

In some aspects, the IL-7 protein is administered to the subject intramuscularly, parenthetically, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. In some aspects, the VEGF antagonist is administered to the subject intravenously, parenthetically, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally. In some aspects, the additional therapeutic agent is administered to the subject parenthetically, intravenously, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally.

In some aspects, the IL-7 protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg. In some aspects, the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg. In some aspects, the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, the IL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks. In some aspects, the IL-7 protein is administered at a dosing frequency of once in eight weeks.

In some aspects, the VEGF antagonist is administered to the subject at a dose of about 0.1 mg/kg to about 20 mg/kg. In some aspects, the VEGF antagonist is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks. In some aspects, the VEGF antagonist is administered at a dosing frequency of once in two weeks.

In some aspects, the anti-VEGF antibody is bevacizumab. In some aspects, the bevacizumab is administered at a dose of about 5 mg/kg to about 15 mg/kg. In some aspects, the bevacizumab is administered at a dose of about 5 mg/kg, about 7.5 mg/kg, about 10 mg/kg, about 12.5 mg/kg, or about 15 mg/kg.

In some aspects, the anti-VEGF antibody is aflibercept. In some aspects, the aflibercept is administered at a dose of about 4 mg/kg to about 15 mg/kg. In some aspects, the aflibercept is administered at a dose of about 4 mg/kg.

### BRIEF DESCRIPTION OF THE FIGURES

FIGS. 1A and 1B show the anti-tumor effects of IL-7 protein and VEGF antagonist combination treatment in a colon adenocarcinoma animal model. FIG. 1A provides a schematic showing the schedule of tumor inoculation and treatment administration. FIG. 1B provides a comparison of tumor volume (mm³) at various time points post tumor inoculation in animals from the different treatment groups. The treatment groups included: (1) IL-7-formulating buffer+isotype control antibody (“Control”); (2) IL-7 protein+isotype control antibody (“IL-7”); (3) IL-7 formulating buffer+VEGF antagonist (e.g., anti-VEGF antibody) (“a-VEGF”); and (4) IL-7 protein+VEGF antagonist (“IL-7+a-VEGF”). The data are shown as mean±S.E.M. GraphPad Prism 9.1.0 software was used for statistical analysis (One-way ANOVA). “**”=p<0.01; and “****”=p<0.0001.

FIG. 2 provides a schematic of the administration schedule of the phase 2 study described in Examples 3 and 4.

FIG. 3 provides a general summary of the phase 2 study described in Examples 3 and 4.

FIGS. 4A and 4B provide interim results of the phase 2 study described in Examples 3 and 4. FIG. 4A shows the current status of the 20 subjects enrolled in the phase 2 study. Specifically, the following status of the subjects are provided: (1) alive (gray bars), (2) dead (dark gray bars), (3) partial response to treatment (black circle), (4) stable disease (black triangle), (5) unconfirmed progression of disease (open square), (6) confirmed progression of disease (black square), and (7) end of treatment (black diamond). FIG. 4B shows absolute lymphocyte count at weeks 0 and 4 after initial administration of the treatment. (*) indicates statistically significant difference between 4 weeks and 0 week (baseline) by Wilcoxan matched-pairs signed rank test.

### DETAILED DESCRIPTION OF THE DISCLOSURE

The present disclosure provides combination regimens comprising an IL-7 protein in combination with a VEGF antagonist, which can be used to treat a tumor. As demonstrated herein, administration of an IL-7 protein in combination with a VEGF antagonist can result in much improved anti-tumor immunity compared to either the IL-7 protein or VEGF antagonist alone. Accordingly, in some aspects, the combination regimens described herein offer an effective and viable treatment options for many cancers. Additional aspects of the present disclosure are provided throughout the present application.

Before the present disclosure is described in greater detail, it is to be understood that this disclosure is not limited to the particular compositions or methods described, which can, of course, vary. As will be apparent to those of skill in the art upon reading this disclosure, each of the individual aspects described and illustrated herein has discrete components and features which can be readily separated from, or combined with, the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Definitions

In order that the present disclosure can be more readily understood, certain terms are first defined. As used in this application, except as otherwise expressly provided herein, each of the following terms shall have the meaning set forth below. Additional definitions are set forth throughout the application.

The term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary of Biochemistry and Molecular Biology, 2nd ed., 2008, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited (e.g., 10), it is to be understood that values that are about the same quantity or amount as the recited value (e.g., ±10%) are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “interleukin-7” or “IL-7” refers to IL-7 polypeptides and derivatives and analogs thereof having substantial amino acid sequence identity to wild-type mature mammalian IL-7s and substantially equivalent biological activity, e.g., in standard bioassays or assays of IL-7 receptor binding affinity. For example, IL-7 refers to an amino acid sequence of a recombinant or non-recombinant polypeptide having an amino acid sequence of: i) a native or naturally-occurring allelic variant of an IL-7 polypeptide, ii) a biologically active fragment of an IL-7 polypeptide, iii) a biologically active polypeptide analog of an IL-7 polypeptide, or iv) a biologically active variant of an IL-7 polypeptide. IL-7 polypeptides of the present disclosure can be obtained from any species, e.g., human, cow or sheep. IL-7 nucleic acid and amino acid sequences are well known in the art. For example, the human IL-7 amino acid sequence has a Genbank accession number of P13232 (SEQ ID NO: 1); the mouse IL-7 amino acid sequence has a Genbank accession number of P10168 (SEQ ID NO: 3); the rat IL-7 amino acid sequence has a Genbank accession number of P56478 (SEQ ID NO: 2); the monkey IL-7 amino acid sequence has a Genbank accession number of NP_001279008 (SEQ ID NO: 4); the cow IL-7 amino acid sequence has a Genbank accession number of P26895 (SEQ ID NO: 5); and the sheep IL-7 amino acid sequence has a Genbank accession number of Q28540 (SEQ ID NO: 6). In some aspects, an IL-7 polypeptide of the present disclosure is a variant of an IL-7 protein. As is apparent from the present disclosure, in some aspects, an IL-7 polypeptide useful for the present does not comprise a signal peptide (see, e.g., Table 1A).

TABLE 1A
Sequences for Exemplary IL-7 Proteins

| | |
|---|---|
| Human IL-7 Protein | MFHVSFRYIFGLPPLILVLLPVASSDCDIEGKDGKQYESVLMVSIDQLLDSMK |
| (SEQ ID NO: 1) | EIGSNCLNNEFNFFKRHICDANKEGMFLFRAARKLRQFLKMNSTGDFDLHLLK |
| (signal peptide | VSEGTTILLNCTGQVKGRKPAALGEAQPTKSLEENKSLKEQKKLNDLCFLKRL |
| bolded) | LQEIKTCWNKILMGTKEH |
| Rat IL-7 Protein | MFHVSFRYIFGIPPLILVLLPVTSSDCHIKDKDGKAFGSVLMISINQLDKMTG |
| (SEQ ID NO: 2) | TDSDCPNNEPNFFKKHLCDDTKEAAFLNRAARKLRQFLKMNISEEFNDHLLRV |
| (signal peptide | SDGTQTLVNCTSKEEKTIKEQKKNDPCFLKRLLREIKTCWNKILKGSI |
| bolded) | |
| Mouse IL-7 Protein | MFHVSFRYIFGIPPLILVLLPVTSSECHIKDKEGKAYESVLMISIDELDKMTG |
| (SEQ ID NO: 3) | TDSNCPNNEPNFFRKHVCDDTKEAAFLNRAARKLKQFLKMNISEEFNVHLLTV |
| (signal peptide | SQGTQTLVNCTSKEEKNVKEQKKNDACFLKRLLREIKTCWNKILKGSI |
| bolded) | |
| Monkey IL-7 Protein | MFHVSFRYIFGLPPLILVLLPVASSDCDIEGKDGKQYESVLMVSIDQLLDSMK |
| (SEQ ID NO: 4) | EIGSNCLNNEFNFFKRHLCDDNKEGMFLFRAARKLKQFLKMNSTGDFDLHLLK |
| (signal peptide | VSEGTTILLNCTGKVKGRKPAALGEPQPTKSLEENKSLKEQKKLNDSCFLKRL |
| bolded) | LQKIKTCWNKILMGTKEH |
| Cow IL-7 Protein | MFHVSFRYIFGIPPLILVLLPVASSDCDISGKDGGAYQNVLMVNIDDLDNMIN |
| (SEQ ID NO: 5) | FDSNCLNNEPNFFKKHSCDDNKEASFLNRASRKLRQFLKMNISDDFKLHLSTV |
| (signal peptide | SQGTLTLLNCTSKGKGRKPPSLSEAQPTKNLEENKSSKEQKKQNDLCFLKILL |
| bolded) | QKIKTCWNKILRGIKEH |
| Sheep IL-7 Protein | MFHVSFRYIFGIPPLILVLLPVASSDCDFSGKDGGAYQNVLMVSIDDLDNMIN |
| (SEQ ID NO: 6) | FDSNCLNNEPNFFKKHSCDDNKEASFLNRAARKLKQFLKMNISDDFKLHLSTV |
| (signal peptide | SQGTLTLLNCTSKGKGRKPPSLGEAQPTKNLEENKSLKEQRKQNDLCFLKILL |
| bolded) | QKIKTCWNKILRGITEH |
| Human IL-7 Protein | DCDIEGKDGKQYESVLMVSIDQLLDSMKEIGSNCLNNEFNFFKRHICDANKEG |
| (SEQ ID NO: 80) | MFLFRAARKLRQFLKMNSTGDFDLHLLKVSEGTTILLNCTGQVKGRKPAALGE |
| (without signal | AQPTKSLEENKSLKEQKKLNDLCFLKRLLQEIKTCWNKILMGTKEH |
| peptide) | |
| Rat IL-7 Protein | DCHIKDKDGKAFGSVLMISINQLDKMTGTDSDCPNNEPNFFKKHL |
| (SEQ ID NO: 81) | CDDTKEAAFLNRAARKLRQFLKMNISEEFNDHLLRVSDGTQTLVNCTSKEEKT |
| (without signal | IKEQKKNDPCFLKRLLR |
| peptide) | EIKTCWNKILKGSI |
| Mouse IL-7 Protein | ECHIKDKEGKAYESVLMISIDELDKMTGTDSNCPNNEPNFFRKHVCDDTKEAA |
| (SEQ ID NO: 82) | FLNRAARKLKQFLKMNISEEFNVHLLTVSQGTQTLVNCTSKEEKNVKEQKKND |
| (without signal | ACFLKRLLREIKTCWNKILKGSI |
| peptide) | |
| Monkey IL-7 Protein | DCDIEGKDGKQYESVLMVSIDQLLDSMKEIGSNCLNNEFNFFKRHLCDDNKEG |
| (SEQ ID NO: 83) | MFLFRAARKLKQFLKMNSTGDFDLHLLKVSEGTTILLNCTGKVKGRKPAALGE |
| (without signal | PQPTKSLEENKSLKEQKKLNDSCFLKRLLQKIKTCWNKILMGTKEH |
| peptide) | |
| Cow IL-7 Protein | DCDISGKDGGAYQNVLMVNIDDLDNMINFDSNCLNNEPNFFKKHSCDDNKEAS |
| (SEQ ID NO: 84) | FLNRASRKLRQFLKMNISDDFKLHLSTVSQGTLTLLNCTSKGKGRKPPSLSEA |
| (without signal | QPTKNLEENKSSKEQKKQNDLCFLKILLQKIKTCWNKILRGIKEH |
| peptide) | |
| Sheep IL-7 Protein | DCDFSGKDGGAYQNVLMVSIDDLDNMINFDSNCLNNEPNFFKKHSCDDNKEAS |
| (SEQ ID NO: 85) | FLNRAARKLKQFLKMNISDDFKLHLSTVSQGTLTLLNCTSKGKGRKPPSLGEA |
| (without signal | QPTKNLEENKSLKEQRKQNDLCFLKILLQKIKTCWNKILRGITEH |
| peptide) | |

As used herein, the term “vascular endothelial growth factor” or “VEGF” refers to proteins produced by many cells and stimulates the formation of blood vessels (i.e., angiogenesis). VEGF is part of a family of proteins comprising five primary members: VEGF-A (also referred to as just “VEGF”), placenta growth factor (PGF), VEGF-B, VEGF-C, and VEGF-D. Unless indicated otherwise, the term “VEGF” refers to the VEGF-A member of the family, including all isoforms (see, e.g., Table 1B) and variants thereof. There are at least 17 known isoforms of human VEGF (i.e., VEGF-A) due to alternative promoter usage, alternative splicing, and/or alternative initiation. Sequences for the different VEGF isoforms are provided in Table 1B (below).

TABLE 1B
Human VEGF Sequences

| Isoform | Description | Sequence |
|---|---|---|
| Isoform | Canonical sequence | MNFLLSWVHWSLALLLYLHHAKWSQAAPMAEGGG |
| VEGF206 | | QNHHEVVKFMDVYQRSYCHPIETLVDIFQEYPDE |
| (UniProt: P15692-1) | | IEYIFKPSCVPLMRCGGCCNDEGLECVPTEESNI |
| | | TMQIMRIKPHQGQHIGEMSFLQHNKCECRPKKDR |
| | | ARQEKKSVRGKGKGQKRKRKKSRYKSWSVYVGAR |
| | | CCLMPWSLPGPHPCGPCSERRKHLFVQDPQTCKC |
| | | SCKNTDSRCKARQLELNERTCRCDKPRR |
| | | (SEQ ID NO: 73) |
| Isoform | Differs from the | MNFLLSWVHWSLALLLYLHHAKWSQAAPMAEGGG |
| VEGF189 | canonical | QNHHEVVKFMDVYQRSYCHPIETLVDIFQEYPDE |
| (UniProt: P15692-2) | sequence as follows: | IEYIFKPSCVPLMRCGGCCNDEGLECVPTEESNI |
| | •166-182: Missing | TMQIMRIKPHQGQHIGEMSFLQHNKCECRPKKDR |
| | | ARQEKKSVRGKGKGQKRKRKKSRYKSWSVPCGPC |
| | | SERRKHLFVQDPQTCKCSCKNTDSRCKARQLELN |
| | | ERTCRCDKPRR |
| | | (SEQ ID NO: 74) |
| Isoform | Differs from the | MNFLLSWVHWSLALLLYLHHAKWSQAAPMAEGGG |
| VEGF183 | canonical | QNHHEVVKFMDVYQRSYCHPIETLVDIFQEYPDE |
| (UniProt: P15692-3) | sequence as follows: | IEYIFKPSCVPLMRCGGCCNDEGLECVPTEESNI |
| | •160-182: Missing | TMQIMRIKPHQGQHIGEMSFLQHNKCECRPKKDR |
| | | ARQEKKSVRGKGKGQKRKRKKSRPCGPCSERRKH |
| | | LFVQDPQTCKCSCKNTDSRCKARQLELNERTCRC |
| | | DKPRR |
| | | (SEQ ID NO: 75) |
| Isoform | Differs from the | MNFLLSWVHWSLALLLYLHHAKWSQAAPMAEGGG |
| VEGF165 | canonical | QNHHEVVKFMDVYQRSYCHPIETLVDIFQEYPDE |
| (UniProt: P15692-4) | sequence as follows: | IEYIFKPSCVPLMRCGGCCNDEGLECVPTEESNI |
| | •141-141: K→N | TMQIMRIKPHQGQHIGEMSFLQHNKCECRPKKDR |
| | •142-182: Missing | ARQENPCGPCSERRKHLFVQDPQTCKCSCKNTDS |
| | | RCKARQLELNERTCRCDKPRR |
| | | (SEQ ID NO: 76) |
| Isoform | Differs from the | MNFLLSWVHWSLALLLYLHHAKWSQAAPMAEGGG |
| VEGF148 | canonical | QNHHEVVKFMDVYQRSYCHPIETLVDIFQEYPDE |
| (UniProt: P15692-5) | sequence as follows: | IEYIFKPSCVPLMRCGGCCNDEGLECVPTEESNI |
| | •141-141: K→N | TMQIMRIKPHQGQHIGEMSFLQHNKCECRPKKDR |
| | •142-182: Missing | ARQENPCGPCSERRKHLFVQDPQTCKCSCKNTDS |
| | •215-215: A→M | RCKM |
| | •216-232: Missing | (SEQ ID NO: 77) |
| Isoform | Differs from the | MNFLLSWVHWSLALLLYLHHAKWSQAAPMAEGGG |
| VEGF145 | canonical | QNHHEVVKFMDVYQRSYCHPIETLVDIFQEYPDE |
| (UniProt: P15692-6) | sequence as follows: | IEYIFKPSCVPLMRCGGCCNDEGLECVPTEESNI |
| | •166-226: Missing | TMQIMRIKPHQGQHIGEMSFLQHNKCECRPKKDR |
| | | ARQEKKSVRGKGKGQKRKRKKSRYKSWSVCDKPR |
| | | R |
| | | (SEQ ID NO: 78) |
| Isoform | Differs from the | MNFLLSWVHWSLALLLYLHHAKWSQAAPMAEGGG |
| VEGF165B | canonical | QNHHEVVKFMDVYQRSYCHPIETLVDIFQEYPDE |
| (UniProt: P15692-8) | sequence as follows: | IEYIFKPSCVPLMRCGGCCNDEGLECVPTEESNI |
| | •141-141: K→N | TMQIMRIKPHQGQHIGEMSFLQHNKCECRPKKDR |
| | •142-182: Missing | ARQENPCGPCSERRKHLFVQDPQTCKCSCKNTDS |
| | •227-232: CDKPRR→ | RCKARQLELNERTCRSLTRKD |
| | SLTRKD | (SEQ ID NO: 79) |
| Isoform | Differs from the | MNFLLSWVHWSLALLLYLHHAKWSQAAPMAEGGG |
| VEGF121 | canonical | QNHHEVVKFMDVYQRSYCHPIETLVDIFQEYPDE |
| (UniProt: P15692-9) | sequence as follows: | IEYIFKPSCVPLMRCGGCCNDEGLECVPTEESNI |
| | •142-226: Missing | TMQIMRIKPHQGQHIGEMSFLQHNKCECRPKKDR |
| | | ARQEKCDKPRR |
| | | (SEQ ID NO: 80) |
| Isoform | Differs from the | MNFLLSWVHWSLALLLYLHHAKWSQAAPMAEGGG |
| VEGF111 | canonical | QNHHEVVKFMDVYQRSYCHPIETLVDIFQEYPDE |
| (UniProt: P15692- | sequence as follows: | IEYIFKPSCVPLMRCGGCCNDEGLECVPTEESNI |
| 10) | •132-226: Missing | TMQIMRIKPHQGQHIGEMSFLQHNKCECRCDKPR |
| | | R |
| | | (SEQ ID NO: 81) |
| Isoform L- | Differs from the | MTDRQTDTAPSPSYHLLPGRRRTVDAAASRGQGP |
| VEGF165 | canonical | EPAPGGGVEGVGARGVALKLFVQLLGCSRFGGAV |
| (UniProt: P15692- | sequence as follows: | VRAGEAEPSGAARSASSGREEPQPEEGEEEEEKE |
| 11) | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •141-141: K→N | QAAPMAEGGGQNHHEVVKFMDVYQRSYCHPIETL |
| | •142-182: Missing | VDIFQEYPDEIEYIFKPSCVPLMRCGGCCNDEGL |
| | Produced by alternative | ECVPTEESNITMQIMRIKPHQGQHIGEMSFLQHN |
| | promoter usage and | KCECRPKKDRARQENPCGPCSERRKHLFVQDPQT |
| | alternative initiation. | CKCSCKNTDSRCKARQLELNERTCRCDKPRR |
| | | (SEQ ID NO: 82) |
| Isoform L- | Differs from the | MTDRQTDTAPSPSYHLLPGRRRTVDAAASRGQGP |
| VEGF121 | canonical | EPAPGGGVEGVGARGVALKLFVQLLGCSRFGGAV |
| (UniProt: P15692- | sequence as follows: | VRAGEAEPSGAARSASSGREEPQPEEGEEEEEKE |
| 12) | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •142-226: Missing | QAAPMAEGGGQNHHEVVKFMDVYQRSYCHPIETL |
| | Produced by alternative | VDIFQEYPDEIEYIFKPSCVPLMRCGGCCNDEGL |
| | promoter usage and | ECVPTEESNITMQIMRIKPHQGQHIGEMSFLQHN |
| | alternative initiation | KCECRPKKDRARQEKCDKPRR |
| | | (SEQ ID NO: 83) |
| Isoform L- | Differs from the | MTDRQTDTAPSPSYHLLPGRRRTVDAAASRGQGP |
| VEGF189 | canonical | EPAPGGGVEGVGARGVALKLFVQLLGCSRFGGAV |
| (UniProt: P15692- | sequence as follows: | VRAGEAEPSGAARSASSGREEPQPEEGEEEEEKE |
| 13) | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •166-182: Missing | QAAPMAEGGGQNHHEVVKFMDVYQRSYCHPIETL |
| | Produced by alternative | VDIFQEYPDEIEYIFKPSCVPLMRCGGCCNDEGL |
| | promoter usage and | ECVPTEESNITMQIMRIKPHQGQHIGEMSFLQHN |
| | alternative initiation. | KCECRPKKDRARQEKKSVRGKGKGQKRKRKKSRY |
| | | KSWSVPCGPCSERRKHLFVQDPQTCKCSCKNTDS |
| | | RCKARQLELNERTCRCDKPRR |
| | | (SEQ ID NO: 84) |
| Isoform L- | Differs from the | MTDRQTDTAPSPSYHLLPGRRRTVDAAASRGQGP |
| VEGF206 | canonical | EPAPGGGVEGVGARGVALKLFVQLLGCSRFGGAV |
| (UniProt: P15692- | sequence as follows: | VRAGEAEPSGAARSASSGREEPQPEEGEEEEEKE |
| 14) | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | Produced by alternative | QAAPMAEGGGQNHHEVVKFMDVYQRSYCHPIETL |
| | promoter usage and | VDIFQEYPDEIEYIFKPSCVPLMRCGGCCNDEGL |
| | alternative initiation | ECVPTEESNITMQIMRIKPHQGQHIGEMSFLQHN |
| | | KCECRPKKDRARQEKKSVRGKGKGQKRKRKKSRY |
| | | KSWSVYVGARCCLMPWSLPGPHPCGPCSERRKHL |
| | | FVQDPQTCKCSCKNTDSRCKARQLELNERTCRCD |
| | | KPRR |
| | | (SEQ ID NO: 85) |
| Isoform #15 | Differs from the | MTDRQTDTAPSPSYHLLPGRRRTVDAAASRGQGP |
| (UniProt: P15692- | canonical | EPAPGGGVEGVGARGVALKLFVQLLGCSRFGGAV |
| 15) | sequence as follows: | VRAGEAEPSGAARSASSGREEPQPEEGEEEEEKE |
| | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •141-182: | QAAPMAEGGGQNHHEVVKFMDVYQRSYCHPIETL |
| | KKSVRGKGKGQK | VDIFQEYPDEIEYIFKPSCVPLMRCGGCCNDEGL |
| | RKRKKSRYKSWS | ECVPTEESNITMQIMRIKPHQGQHIGEMSFLQHN |
| | VYVGARCCLMPW | KCECRPKKDRARQENPCGPCSERRKHLFVQDPQT |
| | SLPGPH→N | CKCSCKNTDSRCKARQLELNERTCRSLTRKD |
| | •227-232: CDKPRR→ | (SEQ ID NO: 86) |
| | SLTRKD | |
| | Starts at an alternative | |
| | upstream CUG codon | |
| Isoform #16 | Differs from the | MTDRQTDTAPSPSYHLLPGRRRTVDAAASRGQGP |
| (UniProt: P15692- | canonical | EPAPGGGVEGVGARGVALKLFVQLLGCSRFGGAV |
| 16) | sequence as follows: | VRAGEAEPSGAARSASSGREEPQPEEGEEEEEKE |
| | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •160-182: Missing | QAAPMAEGGGQNHHEVVKFMDVYQRSYCHPIETL |
| | Starts at an alternative | VDIFQEYPDEIEYIFKPSCVPLMRCGGCCNDEGL |
| | upstream CUG codon. | ECVPTEESNITMQIMRIKPHQGQHIGEMSFLQHN |
| | | KCECRPKKDRARQEKKSVRGKGKGQKRKRKKSRP |
| | | CGPCSERRKHLFVQDPQTCKCSCKNTDSRCKARQ |
| | | LELNERTCRCDKPRR |
| | | (SEQ ID NO: 87) |
| Isoform #17 | Differs from the | MTDRQTDTAPSPSYHLLPGRRRTVDAAASRGQGP |
| (UniProt: P15692- | canonical | EPAPGGGVEGVGARGVALKLFVQLLGCSRFGGAV |
| 17) | sequence as follows: | VRAGEAEPSGAARSASSGREEPQPEEGEEEEEKE |
| | •1-1: M → | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •141-182: | QAAPMAEGGGQNHHEVVKFMDVYQRSYCHPIETL |
| | KKSVRGKGKGQK | VDIFQEYPDEIEYIFKPSCVPLMRCGGCCNDEGL |
| | RKRKKSRYKSWS | ECVPTEESNITMQIMRIKPHQGQHIGEMSFLQHN |
| | VYVGARCCLMPW | KCECRPKKDRARQENPCGPCSERRKHLFVQDPQT |
| | SLPGPH→N | CKCSCKNTDSRCKM |
| | •215-215: A→M | (SEQ ID NO: 88) |
| | •216-232: Missing | |
| | Starts at an alternative | |
| | upstream CUG codon. | |
| Isoform #18 | Differs from the | MTDRQTDTAPSPSYHLLPGRRRTVDAAASRGQGP |
| (UniProt: P15692- | canonical | EPAPGGGVEGVGARGVALKLFVQLLGCSRFGGAV |
| 18) | sequence as follows: | VRAGEAEPSGAARSASSGREEPQPEEGEEEEEKE |
| | •1-1: M→ | EERGPQWRLGARKPGSWTGEAAVCADSAPAARAP |
| | MTDRQTDTAP... | QALARASGRGGRVARRGAEESGPPHSPSRRGSAS |
| | AGPGRASETM | RAGPGRASETMNFLLSWVHWSLALLLYLHHAKWS |
| | •132-226: Missing | QAAPMAEGGGQNHHEVVKFMDVYQRSYCHPIETL |
| | Starts at an alternative | VDIFQEYPDEIEYIFKPSCVPLMRCGGCCNDEGL |
| | upstream CUG codon. | ECVPTEESNITMQIMRIKPHQGQHIGEMSFLQHN |
| | | KCECRCDKPRR |
| | | (SEQ ID NO: 89) |

As used herein, “administering” refers to the physical introduction of a therapeutic agent or a composition comprising a therapeutic agent to a subject, using any of the various methods and delivery systems known to those skilled in the art. The different routes of administration for a therapeutic agent described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrasternal injection and infusion, as well as in vivo electroporation. Alternatively, a therapeutic agent described herein can be administered via a non-parenteral route, such as a topical, epidermal, or mucosal route of administration, for example, intranasally, orally, vaginally, rectally, sublingually, or topically. Administering can also be performed, for example, once, a plurality of times, and/or over one or more extended periods.

The terms “antibody” and “antibodies” are terms of art and can be used interchangeably herein and refer to a molecule with an antigen binding site that specifically binds an antigen. The terms as used to herein include whole antibodies and any antigen binding fragments (i.e., “antigen-binding portions”) or single chains thereof. An “antibody” refers, in one aspect, to a glycoprotein comprising at least two heavy (H) chains and two light (L) chains inter-connected by disulfide bonds, or an antigen-binding portion thereof. In some aspects, an “antibody” refers to a single chain antibody comprising a single variable domain, e.g., VHH domain. Each heavy chain is comprised of a heavy chain variable region (abbreviated herein as VH) and a heavy chain constant region. In some naturally-occurring antibodies, the heavy chain constant region is comprised of three domains, CH1, CH2 and CH3. In some naturally-occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, 10⁻⁸ M or less, 5×10⁻⁹ M or less, or between 10⁻⁸ M and 10⁻¹⁰ M or less, but does not bind with high affinity to unrelated antigens. An antigen is “substantially identical” to a given antigen if it exhibits a high degree of sequence identity to the given antigen, for example, if it exhibits at least 80%, at least 90%, at least 95%, at least 97%, or at least 99% sequence identity to the sequence of the given antigen. By way of example, an antibody that binds specifically to VEGF can, in some aspects, also have cross-reactivity with VEGF antigens from certain primate species (e.g., cynomolgus anti-VEGF antibody), but cannot cross-react with VEGF molecules from other species or with a molecule other than VEGF.

An immunoglobulin can be derived from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. In some aspects, one or more amino acids of the isotype can be mutated to alter effector function. The term “antibody” includes, by way of example, both naturally occurring and non-naturally occurring Abs; monoclonal and polyclonal Abs; chimeric and humanized Abs; human or nonhuman Abs; wholly synthetic Abs; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to VEGF is substantially free of antibodies that bind specifically to antigens other than VEGF). An isolated antibody that binds specifically to VEGF can, however, have cross-reactivity to other antigens, such as VEGF molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The human antibodies of the present disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-PD-1 antibody binds specifically to PD-1 and an anti-VEGF antibody binds specifically to VEGF.

An “antigen-binding portion” of an antibody (also called an “antigen-binding fragment”) refers to one or more fragments of an antibody that retain the ability to bind specifically to the antigen bound by the whole antibody.

As used herein, the terms “specific binding,” “selective binding,” “selectively binds,” and “specifically binds,” refer to antibody binding to an epitope on a predetermined antigen. Typically, the antibody (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE™ 2000 instrument using the predetermined antigen as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen.

The term “naturally-occurring” as used herein as applied to an object refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides. Unless otherwise specified, the terms “protein” and “polypeptide” can be used interchangeably.

The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an antibody is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

For nucleic acids, the term “substantial homology” indicates that two nucleic acids, or designated sequences thereof, when optimally aligned and compared, are identical, with appropriate nucleotide insertions or deletions, in at least about 80% of the nucleotides, at least about 90% to 95%, or at least about 98% to 99.5% of the nucleotides. Alternatively, substantial homology exists when the segments will hybridize under selective hybridization conditions, to the complement of the strand.

For polypeptides, the term “substantial homology” indicates that two polypeptides, or designated sequences thereof, when optimally aligned and compared, are identical, with appropriate amino acid insertions or deletions, in at least about 80% of the amino acids, at least about 90% to 95%, or at least about 98% to 99.5% of the amino acids.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, e.g., as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at worldwideweb.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Some vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, some vectors are capable of directing the expression of genes to which they are operatively linked. Such vectors are referred to herein as “recombinant expression vectors” (or simply, “expression vectors”) In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because some modifications can occur in succeeding generations due to either mutation or environmental influences, such progeny cannot, in fact, be identical to the parent cell, but are still included within the scope of the term “host cell” as used herein.

As used herein, the term “linked” or “conjugated” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

A “cancer” refers a broad group of various diseases characterized by the uncontrolled growth of abnormal cells in the body. Unregulated cell division and growth results in the formation of malignant tumors that invade neighboring tissues and can also metastasize to distant parts of the body through the lymphatic system or bloodstream. “Cancer” as used herein refers to primary, metastatic and recurrent cancers. Non-limiting examples of suitable cancers that can be treated with the present disclosure are provided elsewhere in the present disclosure.

The term “fusion protein” refers to proteins created through the joining of two or more genes that originally coded for separate proteins. Translation of this fusion gene results in a single polypeptide or multiple polypeptides with functional properties derived from each of the original proteins. In some aspects, the two or more genes can comprise a substitution, a deletion, and/or an addition in its nucleotide sequence.

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Various properties of human FcγRs are known in the art. The majority of innate effector cell types coexpress one or more activating FcγR and the inhibitory FcγRIIB, whereas natural killer (NK) cells selectively express one activating Fc receptor (FcγRIII in mice and FcγRIIIA in humans) but not the inhibitory FcγRIIB in mice and humans. Human IgG1 binds to most human Fc receptors and is considered equivalent to murine IgG2a with respect to the types of activating Fc receptors that it binds to.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, an Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains CH2 and CH3 and the hinge between CH1 and CH2 domains. Although the definition of the boundaries of the Fc region of an immunoglobulin heavy chain might vary, as defined herein, the human IgG heavy chain Fc region is defined to stretch from an amino acid residue D221 for IgG1, V222 for IgG2, L221 for IgG3 and P224 for IgG4 to the carboxy-terminus of the heavy chain, wherein the numbering is according to the EU index as in Kabat. The CH2 domain of a human IgG Fc region extends from amino acid 237 to amino acid 340, and the CH3 domain is positioned on C-terminal side of a CH2 domain in an Fc region, i.e., it extends from amino acid 341 to amino acid 447 or 446 (if the C-terminal lysine residue is absent) or 445 (if the C-terminal glycine and lysine residues are absent) of an IgG. As used herein, the Fc region can be a native sequence Fc, including any allotypic variant, or a variant Fc (e.g., a non-naturally occurring Fc). Fc can also refer to this region in isolation or in the context of an Fc-comprising protein polypeptide such as a “binding protein comprising an Fc region,” also referred to as an “Fc fusion protein” (e.g., an antibody or immunoadhesion).

A “native sequence Fc region” or “native sequence Fc” comprises an amino acid sequence that is identical to the amino acid sequence of an Fc region found in nature. Native sequence human Fc regions include a native sequence human IgG1 Fc region; native sequence human IgG2 Fc region; native sequence human IgG3 Fc region; and native sequence human IgG4 Fc region as well as naturally occurring variants thereof. Native sequence Fc include the various allotypes of Fcs (see, e.g., Jefferis et al. (2009) mAbs 1: 1).

Additionally, a Fc (native or variant) of the present disclosure can be in the form of having native sugar chains, increased sugar chains, or decreased sugar chains compared to the native form, or can be in a deglycosylated form. The immunoglobulin Fc sugar chains can be modified by conventional methods such as a chemical method, an enzymatic method, and a genetic engineering method using a microorganism. The removal of sugar chains from an Fc fragment results in a sharp decrease in binding affinity to the C1q part of the first complement component C1, and a decrease or loss of ADCC or CDC, thereby not inducing any unnecessary immune responses in vivo. In this regard, an immunoglobulin Fc region in a deglycosylated or aglycosylated form can be more suitable to the object of the present disclosure as a drug carrier. As used herein, the term “deglycosylation” refers to an Fc region in which sugars are removed enzymatically from an Fc fragment. Additionally, the term “aglycosylation” means that an Fc fragment is produced in an unglycosylated form by a prokaryote, and preferably in E. coli.

As used herein, the term “immune response” refers to a biological response within a vertebrate against foreign agents, which response protects the organism against these agents and diseases caused by them. For instance, the term “anti-tumor immune response” refers to an immune response against a tumor antigen. An immune response is mediated by the action of a cell of the immune system (e.g., a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell or a Th cell, such as a CD4⁺ or CD8⁺ T cell, or the inhibition of a Treg cell.

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease.

The term “effector T cells” (Teff) refers to T cells (e.g., CD4⁺ and CD8⁺ T cells) with cytolytic activities as well as T helper (Th) cells, which secrete cytokines and activate and direct other immune cells, but does not include regulatory T cells (Treg cells). Combination of an IL-7 protein and a VEGF antagonist (e.g., an anti-VEGF antibody) activate and/or increase the frequency of Teff cells, e.g., CD4⁺ and CD8⁺ T cells, in a tumor or blood of a subject.

As used herein, the term “regulatory T cells” (Tregs) refer to a population of T cells with the ability to reduce or suppress the induction and proliferation of effector T cells, and thereby, modulate an immune response. In some aspects, Tregs can suppress an immune response by secreting anti-inflammatory cytokines, such as IL-10, TGF-β, and IL-35, which can interfere with the activation and differentiation of naïve T cells into effector T cells. In some aspects, Tregs can also produce cytolytic molecules, such as Granzyme B, which can induce the apoptosis of effector T cells. In some aspects, the regulatory T cells are natural regulatory T cells (nTregs) (i.e., developed within the thymus). In some aspects, the regulatory T cells are induced regulatory T cells (iTregs) (i.e., naïve T cells that differentiate into Tregs in the peripheral tissue upon exposure to certain stimuli). Methods for identifying Tregs are known in the art. For example, Tregs express certain phenotypic markers (e.g., CD25, Foxp3, or CD39) that can be measured using flow cytometry. See, e.g., International Publication No. WO 2017/062035 A1; Gu J., et al., Cell Mol Immunol 14(6): 521-528 (2017). In some aspects, the Tregs are CD45RA⁻ CD39⁺ T cells.

As used herein, the term “tumor infiltrating lymphocytes” or “TILs” refers to lymphocytes (e.g., effector T cells) that have migrated from the periphery (e.g., from the blood) into a tumor. In some aspects, the tumor infiltrating lymphocytes are CD4+ TILs. In some aspects, the tumor infiltrating lymphocytes are CD8+ TILs.

An increased ability to stimulate an immune response or the immune system, can result from an enhanced agonist activity of T cell costimulatory receptors and/or an enhanced antagonist activity of inhibitory receptors. An increased ability to stimulate an immune response or the immune system can be reflected by a fold increase of the EC50 or maximal level of activity in an assay that measures an immune response, e.g., an assay that measures changes in cytokine or chemokine release, cytolytic activity (determined directly on target cells or indirectly via detecting CD107a or granzymes) and proliferation. The ability to stimulate an immune response or the immune system activity can be enhanced by at least 10%, 30%, 50%, 75%, 2 fold, 3 fold, 5 fold or more.

A “variant” of an, e.g., IL-7 protein, is defined as an amino acid sequence that is altered by one or more amino acids. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues can be substituted, inserted or deleted without abolishing biological activity can be found using computer programs well known in the art, for example software for molecular modeling or for producing alignments. For example, the variant IL-7 proteins included within the disclosure include IL-7 proteins that retain IL-7 activity. Similarly, variants of a VEGF antagonist described herein refer to those that maintain antagonistic activity against VEGF. Variants which also include additions, substitutions or deletions are also included within the present disclosure as long as the variants retain substantially equivalent biological activity of the non-variant (e.g., wild-type) counterpart. For example, truncations of IL-7 which retain comparable biological activity as the full length form of the IL-7 protein are included within the present disclosure. The activity of a therapeutic agent described herein (e.g., an IL-7 protein and/or a VEGF antagonist) can be measured using any suitable methods known in the art.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino acid or nucleic acid sequence). The percent identity between the two sequences is a function of the number of identical positions shared by the sequences (i.e., % homology=# of identical positions/total # of positions.times.100). The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the NBLAST and XBLAST programs of Altschul, et al., (1990) J. Mol. Biol. 215:403-10. BLAST nucleotide searches can be performed with the NBLAST program, score=100, wordlength=12. BLAST protein searches can be performed with the XBLAST program, score=50, wordlength=3. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used.

A “subject” includes any human or nonhuman animal. The term “nonhuman animal” includes, but is not limited to, vertebrates such as nonhuman primates, sheep, dogs, and rodents such as mice, rats and guinea pigs. In some aspects, the subject is a human. The terms “subject” and “patient” are used interchangeably herein.

The term “therapeutically effective amount” or “therapeutically effective dosage” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In some aspects, a “therapeutically effective amount” is the amount of IL-7 protein and the amount of a VEGF antagonist (e.g., anti-VEGF antibody), in combination, clinically proven to affect a significant decrease in cancer or slowing of progression (regression) of cancer, such as an advanced solid tumor. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “dosing frequency” refers to the number of times a therapeutic agent (e.g., an IL-7 protein and/or a VEGF antagonist) is administered to a subject within a specific time period. Dosing frequency can be indicated as the number of doses per a given time, for example, once per day, once a week, or once in two weeks. As used herein, “dosing frequency” is applicable where a subject receives multiple (or repeated) administrations of a therapeutic agent. Suitable dosing frequency for any of the IL-7 proteins and VEGF antagonists described herein are provided elsewhere in the present disclosure.

As used herein, the term “standard of care” refers to a treatment that is accepted by medical experts as a proper treatment for a certain type of disease and that is widely used by healthcare professionals. The term can be used interchangeable with any of the following terms: “best practice,” “standard medical care,” and “standard therapy.” As further described elsewhere in the present disclosure, in some aspects, a standard of care comprises a chemotherapy (e.g., temozolomide). In some aspects, a standard of care comprises a radiotherapy. In some aspects, a standard of care comprises both radiotherapy and chemotherapy (e.g., temozolomide).

As used herein, the term “drug” refers to any bioactive agent (e.g., an IL-7 protein and/or a VEGF antagonist) intended for administration to a human or non-human mammal to prevent or treat a disease or other undesirable condition. Drugs include hormones, growth factors, proteins, peptides and other compounds. In some aspects, a drug disclosed herein is an anti-cancer agent. Unless indicated otherwise, the term “drug” can be used interchangeably with the term “therapeutic agent.”

By way of example, an “anti-cancer agent” promotes cancer regression in a subject or prevents further tumor growth. In some aspects, a therapeutically effective amount of the drug promotes cancer regression to the point of eliminating the cancer. “Promoting cancer regression” means that administering an effective amount of the drug, alone or in combination with an anti-neoplastic agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the drug to promote cancer regression in the patient. Physiological safety refers to the level of toxicity, or other adverse physiological effects at the cellular, organ and/or organism level (adverse effects) resulting from administration of the drug.

By way of example for the treatment of tumors, a therapeutically effective amount of an anti-cancer agent can inhibit cell growth or tumor growth by at least about 10%, at least about 20%, by at least about 40%, by at least about 60%, or by at least about 80% relative to untreated subjects or, in some aspects, relative to patients treated with a standard-of-care therapy. In some aspects, tumor regression can be observed and continue for a period of at least about 20 days, at least about 40 days, or at least about 60 days. Notwithstanding these ultimate measurements of therapeutic effectiveness, evaluation of immunotherapeutic drugs must also make allowance for “immune-related” response patterns.

As used herein, the term “immune checkpoint inhibitor” refers to molecules that totally or partially reduce, inhibit, interfere with or modulate one or more checkpoint proteins. Checkpoint proteins regulate T-cell activation or function. Numerous checkpoint proteins are known, such as CTLA-4 and its ligands CD80 and CD86; and PD-1 with its ligands PD-L1 and PD-L2. Pardoll, D. M., Nat Rev Cancer 12(4):252-64 (2012). These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies or are derived from antibodies. Suitable immune checkpoint inhibitors that are useful for the present disclosure are provided elsewhere in the present disclosure.

As used herein, the term “reference subject” refers to a corresponding subject (e.g., a cancer subject) who did not receive a combination treatment described herein (e.g., an IL-7 protein in combination with a VEGF antagonist). In some aspects, a reference subject refers to a corresponding subject who received the IL-7 protein alone or the VEGF antagonist alone. In some aspects, the reference subject received neither the IL-7 protein nor the VEGF antagonist. The term “reference subject” can also refer to a same subject (i.e., treated with the methods provided herein) but prior to the administration of a combination of IL-7 protein and a VEGF antagonist. In some aspects, the term “reference subject” refers to an average of a population of subjects (e.g., cancer subjects).

As used herein, the terms “ug” and “uM” are used interchangeably with “μg” and “μM,” respectively.

Various aspects described herein are described in further detail in the following subsections.

### II. Methods of the Disclosure

Provided herein is a method of treating a tumor (or a cancer) in a subject in need thereof, comprising administering to the subject an interleukin-7 (IL-7) protein in combination with an antagonist of vascular endothelial growth factor (“VEGF”) (“VEGF antagonist”). Non-limiting examples of IL-7 proteins and VEGF antagonists that are useful for the methods are provided elsewhere in the present disclosure. As demonstrated herein, a treatment regimen comprising an IL-7 protein and a VEGF antagonist (such as those described herein) can be associated with much improved anti-tumor immunity compared to either therapeutic agent alone.

Accordingly, in some aspects, administering a combination treatment described herein (e.g., an IL-7 protein in combination with a VEGF antagonist) can increase an anti-tumor immune response in a subject. In some aspects, after the administration, the anti-tumor immune response is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to the corresponding value in a reference subject. For instance, in some aspects, the anti-tumor immune response in the subject is increased compared to the anti-tumor immune response in the subject prior to the administration of the combination treatment. In some aspects, the anti-tumor immune response in the subject is increased compared to a corresponding subject that received an administration of the IL-7 protein alone or the VEGF antagonist alone. In some aspects, the anti-tumor immune response in the subject is increased compared to a corresponding subject that received neither the IL-7 protein nor the VEGF antagonist.

In some aspects, an increased anti-tumor immune response can be associated with one or more of the following therapeutic effects: (i) reduced tumor volume and/or growth; (ii) increased effector activity (e.g., ability to kill a tumor cell) of a tumor-specific T cell; (iii) increased number of tumor-specific effector T cells, e.g., in a tumor; (iv) increased number of tumor-infiltrating lymphocytes (TILs), e.g., in a tumor; (v) decreased number of myeloid-derived suppressor cells (MDSCs), e.g., in a tumor; (vi) decreased number of regulatory T cells (Tregs), e.g., in a tumor; (vii) increased duration of survival of the subject; (viii) decreased expression of TOX on CD8+ T cells, e.g., in a tumor, tumor-draining lymph node (TDLN), or both; (ix) increased number of stem-like T cells, e.g., in a tumor, TDLN, or both; or (x) any combination thereof. Unless indicated otherwise, the term “number” refers to both absolute number as well as percentage. Any of the therapeutic effects described above or elsewhere in the present disclosure can be determined using any of the suitable methods provided herein and/or known in the art.

In some aspects, administering a combination treatment described herein (e.g., an IL-7 protein in combination with a VEGF antagonist) reduces a tumor volume in the subject. In some aspects, the tumor volume is reduced by at least about 5%, at least about 10%, at least about 20%, at least about 30%, at least about 40%, at least about 50%, at least about 60%, at least about 70%, at least about 80%, at least about 90%, or at least about 100% compared to a corresponding value in a reference subject. In some aspects, administering a combination treatment described herein (e.g., an IL-7 protein in combination with a VEGF antagonist) can reduce the growth of a tumor (“tumor growth”) in a subject. In some aspects, the tumor growth is reduced by at least about 5%, at least about 10%, at least about 20%, at least about 30%, at least about 40%, at least about 50%, at least about 60%, at least about 70%, at least about 80%, at least about 90%, or at least about 100% compared to a corresponding value in a reference subject.

In some aspects, administering a combination treatment described herein (e.g., an IL-7 protein in combination with a VEGF antagonist) can increase an effector activity of a tumor-specific T cell (e.g., CD8⁺ T cell and/or CD4⁺ T cell) in the subject. In some aspects, the effector activity is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject. In some aspects, the effector activity comprises any cytolytic activity of a T cell (e.g., CD8⁺ T cell and/or CD4⁺ T cell) that can be useful in the treatment of a cancer. Non-limiting examples of such effector activity include the ability to produce effector molecules (e.g., cytokines, perforin, and/or granzyme) upon antigen stimulation; ability to recognize and kill tumor cells; or both.

In some aspects, administering a combination treatment described herein (e.g., an IL-7 protein in combination with a VEGF antagonist) can increase the number of tumor-specific effector T cells (e.g., CD8+ and/or CD4+) in a subject. In some aspects, the number of tumor-specific effector T cells is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject.

Not to be bound by any one theory, in some aspects, the increased number is due to increased proliferation of the tumor-specific effector T cells. Accordingly, in some aspects, after the administration of a combination treatment described herein (e.g., an IL-7 protein in combination with a VEGF antagonist), the proliferation of the tumor-specific effector T cells is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject. In some aspects, the increase in the number of tumor-specific effector T cells is due to the ability of the T cells to survive and/or persist. In some aspects, after the administration, the ability of the tumor-specific effector T cells to survive and/or persist in the subject is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject. In some aspects, a combination treatment described herein (e.g., an IL-7 protein in combination with a VEGF antagonist), when administered to a subject, can increase both the proliferation and survival/persistence of the tumor-specific effector T cells.

In some aspects, the tumor-specific effector T cells are tumor-infiltrating lymphocytes (TILs). Accordingly, in some aspects, administering a combination treatment described herein (e.g., an IL-7 protein in combination with a VEGF antagonist) can increase the number of TILs in a tumor of a subject. In some aspects, the number of TILs in a tumor of the subject is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject. In some aspects, the increased number of TILs in a tumor of the subject can be associated with an increased recruitment of the TILs from the periphery to the tumor. In some aspects, after the administration of a combination treatment described herein (e.g., an IL-7 protein in combination with a VEGF antagonist), the recruitment of the TILs into the tumor is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject.

In some aspects, administering a combination treatment described herein (e.g., an IL-7 protein in combination with a VEGF antagonist) can reduce the number of regulatory T cells (Tregs) in a tumor of a subject. In some aspects, the regulatory T cells are CD4⁺ regulatory T cells. In some aspects, the regulatory T cells are Foxp3*. In some aspects, the number and/or percentage of regulatory T cells in a tumor is decreased by at least about 5%, at least about 10%, at least about 20%, at least about 30%, at least about 40%, at least about 50%, at least about 60%, at least about 70%, at least about 80%, at least about 90%, or about 100% compared to a corresponding value in a reference subject.

In some aspects, administering a combination treatment described herein (e.g., an IL-7 protein in combination with a VEGF antagonist) can decrease the number of myeloid-derived suppressor cells (MDSCs) in a tumor of a subject. As used herein, the term “myeloid-derived suppressor cells” (MDSCs) refer to a heterogeneous population of immune cells that are defined by their myeloid origin, immature state, and ability to potently suppress T cell responses. They are known to expand in certain pathological conditions, such as chronic infections and cancers. In some aspects, the MDSCs are monocytic MDSCs (M-MDSCs). In some aspects, the MDSCs are polymorphonuclear MDSCs (PMN-MDSCs). In some aspects, after the administration, the number of MDSCs in the tumor is decreased by at least about 5%, at least about 10%, at least about 20%, at least about 30%, at least about 40%, at least about 50%, at least about 60%, at least about 70%, at least about 80%, at least about 90%, or about 100% compared to a corresponding value in a reference subject.

As will be apparent to those skilled in the arts, in some aspects, one or more of the therapeutic effects described above can lead to (i) increased survival (e.g., progression-free survival) of the subject, (ii) increased response rate, (iii) decreased progression of the disease, or (iv) a combination thereof. Accordingly, in some aspects, administering a combination treatment described herein (e.g., an IL-7 protein in combination with a VEGF antagonist) can increase the duration of survival of a subject (e.g., afflicted with a tumor). In some aspects, the duration of survival is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject. In some aspects, after the administration, duration of survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year or more when compared to a reference subject. In some aspects, after the administration, the duration of survival of the subject is increased at a level higher than (about one month higher than, about two months higher than, about three months higher than, about four months higher than, about five months higher than, about six months higher than, about seven months higher than, about eight months higher than, about nine months higher than, about ten months higher than, about eleven months higher than, or about one year higher than) the duration of survival of a reference subject.

In some aspects, administering a combination treatment described herein (e.g., an IL-7 protein in combination with a VEGF antagonist) can increase the duration of progression-free survival of a subject (e.g., cancer patient). For example, the progression free survival of the subject is increased by at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 1 year when compared to a reference subject.

In some aspects, administering a combination treatment described herein (e.g., an IL-7 protein in combination with a VEGF antagonist) can increase the response rate in a group of subjects. For example, the response rate in a group of subjects is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject.

In some aspects, administering a combination treatment described herein (e.g., an IL-7 protein in combination with a VEGF antagonist) can decrease the progression of the cancer. In some aspects, after the administration, the progression of the disease is reduced by at least about 5%, at least about 10%, at least about 20%, at least about 30%, at least about 40%, at least about 50%, at least about 60%, at least about 70%, at least about 80%, at least about 90%, or about 100% compared to a corresponding value in a reference subject.

In some aspects, administering a combination treatment described herein (e.g., an IL-7 protein in combination with a VEGF antagonist) can decrease the expression of TOX in CD8+ T cells, e.g., present in the tumor, tumor-draining lymph node, or both. In some aspects, after the administration, the expression of TOX in the CD8+ T cells is decreased by at least about 5%, at least about 10%, at least about 20%, at least about 30%, at least about 40%, at least about 50%, at least about 60%, at least about 70%, at least about 80%, at least about 90%, or at least about 100% compared to a corresponding value in a reference subject. Not to be bound by any one theory, at least on CD8+ T cells, TOX is thought to play an important role in the induction of T cell exhaustion. See, e.g., Khan et al., Nature 571: 211-218 (2019). Accordingly, in some aspects, a combination treatment described herein (e.g., an IL-7 protein in combination with a VEGF antagonist) can help reduce T cell exhaustion when administered to a subject.

In some aspects, administering a combination treatment described herein can increase the number of stem-like T cells, e.g., present in the tumor, tumor-draining lymph node, or both. As used herein, “stem-like T cells” refer to T cells that are less-differentiated while retaining effector activity (e.g., ability to kill tumor cells). Stem-like T cells can be identified by their phenotypic expression using any suitable methods known in the art (e.g., flow cytometry). In some aspects, stem-like T cells useful for the present disclosure are TCF1+ and TOX-. In some aspects, the stem-like T cells are additionally TIM3-. In some aspects, after the administration, the number of stem like T cells is increased by at least about 0.5-fold, at least about 1-fold, at least about 2-fold, at least about 3-fold, at least about 4-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 40-fold, or at least about 50-fold compared to a corresponding value in a reference subject.

In some aspects, the subject being treated in the method is a nonhuman animal such as a rat or a mouse. In some aspects, the subject being treated in the method is a human.

As will be apparent from the present disclosure, combination therapies described herein (e.g., an IL-7 protein in combination with a VEGF antagonist) can be used to treat any suitable cancers known in the art. Non-limiting examples of cancers (or tumors) that can be treated with the disclosures provided herein include squamous cell carcinoma, small-cell lung cancer (SCLC), non-small cell lung cancer, squamous non-small cell lung cancer (NSCLC), nonsquamous NSCLC, gastrointestinal cancer, renal cancer (e.g., clear cell carcinoma), ovarian cancer, liver cancer (e.g., hepatocellular carcinoma), colorectal cancer, endometrial cancer, kidney cancer (e.g., renal cell carcinoma (RCC)), prostate cancer (e.g., hormone refractory prostate adenocarcinoma), thyroid cancer, pancreatic cancer, cervical cancer, stomach cancer, bladder cancer, hepatoma, breast cancer, colon carcinoma, and head and neck cancer (or carcinoma), gastric cancer, germ cell tumor, pediatric sarcoma, sinonasal natural killer, melanoma (e.g., metastatic malignant melanoma, such as cutaneous or intraocular malignant melanoma), bone cancer, skin cancer, uterine cancer, cancer of the anal region, testicular cancer, carcinoma of the fallopian tubes, carcinoma of the endometrium, carcinoma of the cervix, carcinoma of the vagina, carcinoma of the vulva, cancer of the esophagus (e.g., gastroesophageal junction cancer), cancer of the small intestine, cancer of the endocrine system, cancer of the parathyroid gland, cancer of the adrenal gland, sarcoma of soft tissue, cancer of the urethra, cancer of the penis, solid tumors of childhood, cancer of the ureter, carcinoma of the renal pelvis, tumor angiogenesis, pituitary adenoma, Kaposi's sarcoma, epidermoid cancer, squamous cell cancer, T-cell lymphoma, environmentally-induced cancers including those induced by asbestos, virus-related cancers or cancers of viral origin (e.g., human papilloma virus (HPV-related or -originating tumors)), and hematologic malignancies derived from either of the two major blood cell lineages, i.e., the myeloid cell line (which produces granulocytes, erythrocytes, thrombocytes, macrophages and mast cells) or lymphoid cell line (which produces B, T, NK and plasma cells), such as all types of leukemias, lymphomas, and myelomas, e.g., acute, chronic, lymphocytic and/or myelogenous leukemias, such as acute leukemia (ALL), acute myelogenous leukemia (AML), chronic lymphocytic leukemia (CLL), and chronic myelogenous leukemia (CML), undifferentiated AML (MO), myeloblastic leukemia (Ml), myeloblastic leukemia (M2; with cell maturation), promyelocytic leukemia (M3 or M3 variant [M3V]), myelomonocytic leukemia (M4 or M4 variant with eosinophilia [M4E]), monocytic leukemia (M5), erythroleukemia (M6), megakaryoblastic leukemia (M7), isolated granulocytic sarcoma, and chloroma; lymphomas, such as Hodgkin's lymphoma (HL), non-Hodgkin's lymphoma (NHL), B cell hematologic malignancy, e.g., B-cell lymphomas, T-cell lymphomas, lymphoplasmacytoid lymphoma, monocytoid B-cell lymphoma, mucosa-associated lymphoid tissue (MALT) lymphoma, anaplastic (e.g., Ki1⁺) large-cell lymphoma, adult T-cell lymphoma/leukemia, mantle cell lymphoma, angio immunoblastic T-cell lymphoma, angiocentric lymphoma, intestinal T-cell lymphoma, primary mediastinal B-cell lymphoma, precursor T-lymphoblastic lymphoma, T-lymphoblastic; and lymphoma/leukemia (T-Lbly/T-ALL), peripheral T-cell lymphoma, lymphoblastic lymphoma, post-transplantation lymphoproliferative disorder, true histiocytic lymphoma, primary effusion lymphoma, B cell lymphoma, lymphoblastic lymphoma (LBL), hematopoietic tumors of lymphoid lineage, acute lymphoblastic leukemia, diffuse large B-cell lymphoma, Burkitt's lymphoma, follicular lymphoma, diffuse histiocytic lymphoma (DHL), immunoblastic large cell lymphoma, precursor B-lymphoblastic lymphoma, cutaneous T-cell lymphoma (CTLC) (also called mycosis fungoides or Sezary syndrome), and lymphoplasmacytoid lymphoma (LPL) with Waldenstrom's macroglobulinemia; myelomas, such as IgG myeloma, light chain myeloma, nonsecretory myeloma, smoldering myeloma (also called indolent myeloma), solitary plasmocytoma, and multiple myelomas, chronic lymphocytic leukemia (CLL), hairy cell lymphoma; hematopoietic tumors of myeloid lineage, tumors of mesenchymal origin, including fibrosarcoma and rhabdomyoscarcoma; seminoma, teratocarcinoma, tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma, hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) of the T-cell type; a/d T-NHL hepatosplenic lymphoma; peripheral/post-thymic T cell lymphoma (pleomorphic and immunoblastic subtypes); angiocentric (nasal) T-cell lymphoma; cancer of the head or neck, renal cancer, rectal cancer, cancer of the thyroid gland; acute myeloid lymphoma, and any combinations thereof.

In some aspects, a cancer (or tumor) that can be treated comprises a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, carcinoma, sarcoma, leukemia, lymphoma, myeloma, or a combination thereof. In some aspects, a cancer (tumor) that can be treated with the present disclosure comprises a pancreatic cancer. In some aspects, a cancer (tumor) that can be treated comprises a brain cancer. In some aspects, brain cancer comprise a glioma. In some aspects, the brain cancer comprises a glioblastoma. In some aspects, glioblastoma comprises primary glioblastoma. In some aspects, glioblastoma comprises secondary glioblastoma. In some aspects, a glioblastoma that can be treated with the present disclosure is refractory (e.g., to previous cancer therapy, e.g., a standard of care comprising radiotherapy and/or chemotherapy such as temozolomide). In some aspects, a cancer (tumor) that can be treated comprises a skin cancer. Non-limiting examples of skin cancers include: a Merkel cell carcinoma (MCC), basal cell carcinoma (BCC), cutaneous squamous cell carcinoma (cSCC), melanoma, or a combination thereof.

In some aspects, the disclosures provided herein (e.g., treatment comprising an IL-7 protein in combination with a VEGF antagonist) can also be used for treatment of metastatic cancers, unresectable, refractory cancers (e.g., cancers refractory to previous cancer therapy, e.g., immunotherapy and/or standard of care), and/or recurrent cancers. In some aspects, the previous cancer therapy comprises a chemotherapy. In some aspects, the chemotherapy comprises a platinum-based therapy. In some aspects, the platinum-based therapy comprises a platinum-based antineoplastic selected from the group consisting of cisplatin, carboplatin, oxaliplatin, nedaplatin, triplatin tetranitrate, phenanthriplatin, picoplatin, satraplatin, and any combination thereof. In some aspects, the platinum-based therapy comprises cisplatin. In some aspects, the platinum-based therapy comprises carboplatin.

In some aspects, a subject to be treated with disclosures provided herein has received one, two, three, four, five or more prior cancer treatments. In some aspects, the subject is treatment-naïve (i.e., has never received a prior cancer treatment). In some aspects, the subject has progressed on other cancer treatments. In some aspects, the prior cancer treatment comprised an immunotherapy. In some aspects, the prior cancer treatment comprised a chemotherapy. In some aspects, the prior cancer treatment comprised a radiotherapy. In some aspects, the prior cancer treatment comprised a standard of care, e.g., chemotherapy (e.g., temozolomide) and/or radiotherapy. In some aspects, the tumor has reoccurred. In some aspects, the tumor is metastatic. In some aspects, the tumor is not metastatic. Accordingly, as further described elsewhere in the present disclosure, in some aspects, a subject to be treated with the present disclosure suffers from a cancer which is refractory to a prior cancer treatment (e.g., standard of care). In some aspects, a subject that can be treated with the present disclosure suffers from a glioblastoma which is refractory to a standard of care (e.g., radiotherapy and/or temozolomide).

In some aspects, the unit dose (e.g., for human use) of an IL-7 protein disclosed herein can be in the range of about 0.001 mg/kg to about 10 mg/kg. In some aspects, the unit dose of an IL-7 protein is in the range of about 0.01 mg/kg to about 2 mg/kg. In some aspects, the unit dose is in the range of about 0.02 mg/kg to about 1 mg/kg.

In some aspects, an IL-7 protein disclosed herein can be administered to a subject at a weight-based dose. In some aspects, an IL-7 protein can be administered at a weight-based dose between about 20 μg/kg and about 600 μg/kg. In some aspects, an IL-7 protein of the present disclosure can be administered at a weight-based dose of about 20 μg/kg, about 60 μg/kg, about 120 μg/kg, about 240 μg/kg, about 360 μg/kg, about 480 μg/kg, or about 600 μg/kg. In some aspects, the IL-7 protein is administered to the subject at a dose of 60 μg/kg. In some aspects, the IL-7 protein is administered to the subject at a dose of 120 μg/kg. In some aspects, the IL-7 protein is administered to the subject at a dose of 240 μg/kg.

In some aspects, an IL-7 protein disclosed herein can be administered to a subject at a dose greater than about 600 μg/kg. In some aspects, an IL-7 protein is administered to a subject at a dose greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

In some aspects, an IL-7 protein of the present disclosure is administered at a dose of between 610 μg/kg and about 1,200 μg/kg, between 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

In some aspects, an IL-7 protein of the present disclosure is administered at a dose of between 610 μg/kg and about 1,200 μg/kg. In some aspects, an IL-7 protein is administered at a dose of between 650 μg/kg and about 1,200 μg/kg. In some aspects, an IL-7 protein is administered at a dose of between about 700 μg/kg and about 1,200 μg/kg. In some aspects, an IL-7 protein is administered at a dose of between about 750 μg/kg and about 1,200 μg/kg. In some aspects, an IL-7 protein is administered at a dose of between about 800 μg/kg and about 1,200 μg/kg. In some aspects, an IL-7 protein is administered at a dose of between about 850 μg/kg and about 1,200 μg/kg. In some aspects, an IL-7 protein is administered at a dose of between about 900 μg/kg and about 1,200 μg/kg. In some aspects, an IL-7 protein is administered at a dose of between about 950 μg/kg and about 1,200 μg/kg. In some aspects, an IL-7 protein disclosed herein is administered at a dose of between about 1,000 μg/kg and about 1,200 μg/kg. In some aspects, an IL-7 protein is administered at a dose of between about 1,050 μg/kg and about 1,200 μg/kg. In some aspects, an IL-7 protein is administered at a dose of between about 1,100 μg/kg and about 1,200 μg/kg. In some aspects, an IL-7 protein is administered at a dose of between about 1,200 μg/kg and about 2,000 μg/kg. In some aspects, an IL-7 protein is administered at a dose of between about 1,300 μg/kg and about 2,000 μg/kg. In some aspects, an IL-7 protein is administered at a dose of between about 1,500 μg/kg and about 2,000 μg/kg. In some aspects, an IL-7 protein is administered at a dose of between about 1,700 μg/kg and about 2,000 μg/kg. In some aspects, an IL-7 protein is administered at a dose of between about 610 μg/kg and about 1,000 μg/kg. In some aspects, an IL-7 protein is administered at a dose of between about 650 μg/kg and about 1,000 μg/kg. In some aspects, an IL-7 protein is administered at a dose of between about 700 μg/kg and about 1,000 μg/kg. In some aspects, an IL-7 protein is administered at a dose of between about 750 μg/kg and about 1,000 μg/kg. In some aspects, an IL-7 protein is administered at a dose of between about 800 μg/kg and about 1,000 μg/kg. In some aspects, an IL-7 protein is administered at a dose of between about 850 μg/kg and about 1,000 μg/kg. In some aspects, an IL-7 protein of the present disclosure is administered at a dose of between about 900 μg/kg and about 1,000 μg/kg. In some aspects, an IL-7 protein is administered at a dose of between about 950 μg/kg and about 1,000 μg/kg.

In some aspects, an IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg. In some aspects, an IL-7 protein is administered at a dose of between about 700 μg/kg and about 900 μg/kg. In some aspects, an IL-7 protein is administered at a dose of between about 750 μg/kg and about 950 μg/kg. In some aspects, an IL-7 protein is administered at a dose of between about 700 μg/kg and about 850 μg/kg. In some aspects, an IL-7 protein is administered at a dose of between about 750 μg/kg and about 850 μg/kg. In some aspects, an IL-7 protein is administered at a dose of between about 700 μg/kg and about 800 μg/kg. In some aspects, an IL-7 protein is administered at a dose of between about 800 μg/kg and about 900 μg/kg. In some aspects, an IL-7 protein is administered at a dose of between about 750 μg/kg and about 850 μg/kg. In some aspects, an IL-7 protein is administered at a dose of between about 850 μg/kg and about 950 μg/kg.

In some aspects, an IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

In some aspects, an IL-7 protein is administered at a dose of about 650 μg/kg. In some aspects, an IL-7 protein disclosed herein is administered at a dose of about 680 μg/kg. In some aspects, an IL-7 protein is administered at a dose of about 700 μg/kg. In some aspects, an IL-7 protein is administered at a dose of about 720 μg/kg. In some aspects, an IL-7 protein is administered at a dose of about 740 μg/kg. In some aspects, an IL-7 protein is administered at a dose of about 750 μg/kg. In some aspects, an IL-7 protein is administered at a dose of about 760 μg/kg. In some aspects, an IL-7 protein is administered at a dose of about 780 μg/kg. In some aspects, an IL-7 protein is administered at a dose of about 800 μg/kg. In some aspects, an IL-7 protein is administered at a dose of about 820 μg/kg. In some aspects, an IL-7 protein is administered at a dose of about 840 μg/kg. In some aspects, an IL-7 protein is administered at a dose of about 850 μg/kg. In some aspects, an IL-7 protein is administered at a dose of about 860 μg/kg. In some aspects, an IL-7 protein is administered at a dose of about 880 μg/kg. In some aspects, an IL-7 protein is administered at a dose of about 900 μg/kg. In some aspects, an IL-7 protein is administered at a dose of about 920 μg/kg. In some aspects, an IL-7 protein is administered at a dose of about 940 μg/kg. In some aspects, an IL-7 protein is administered at a dose of about 950 μg/kg. In some aspects, an IL-7 protein is administered at a dose of about 960 μg/kg. In some aspects, an IL-7 protein is administered at a dose of about 980 μg/kg. In some aspects, an IL-7 protein is administered at a dose of about 1,000 μg/kg. In some aspects, an IL-7 protein is administered at a dose of about 1,020 μg/kg. In some aspects, an IL-7 protein is administered at a dose of about 1,040 μg/kg. In some aspects, an IL-7 protein is administered at a dose of about 1,060 μg/kg. In some aspects, an IL-7 protein is administered at a dose of about 1,080 μg/kg. In some aspects, an IL-7 protein is administered at a dose of about 1,100 μg/kg. In some aspects, an IL-7 protein is administered at a dose of about 1,120 μg/kg. In some aspects, an IL-7 protein is administered at a dose of about 1,140 μg/kg. In some aspects, an IL-7 protein is administered at a dose of about 1,160 μg/kg. In some aspects, an IL-7 protein is administered at a dose of about 1,180 μg/kg. In some aspects, an IL-7 protein is administered at a dose of about 1,200 μg/kg. In some aspects, an IL-7 protein is administered at a dose of about 1,220 μg/kg. In some aspects, an IL-7 protein is administered at a dose of about 1,240 μg/kg. In some aspects, an IL-7 protein is administered at a dose of about 1,260 μg/kg. In some aspects, an IL-7 protein is administered at a dose of about 1,280 μg/kg. In some aspects, an IL-7 protein is administered at a dose of about 1,300 μg/kg. In some aspects, an IL-7 protein is administered at a dose of about 1,320 μg/kg. In some aspects, an IL-7 protein is administered at a dose of about 1,340 μg/kg. In some aspects, an IL-7 protein is administered at a dose of about 1,360 μg/kg. In some aspects, an IL-7 protein is administered at a dose of about 1,380 μg/kg. In some aspects, an IL-7 protein is administered at a dose of about 1,400 μg/kg. In some aspects, an IL-7 protein is administered at a dose of about 1,420 μg/kg. In some aspects, an IL-7 protein is administered at a dose of about 1,440 μg/kg. In some aspects, an IL-7 protein is administered at a dose of about 1,460 μg/kg. In some aspects, an IL-7 protein is administered at a dose of about 1,480 μg/kg. In some aspects, an IL-7 protein is administered at a dose of about 1,500 μg/kg. In some aspects, an IL-7 protein is administered at a dose of about 1,520 μg/kg. In some aspects, an IL-7 protein is administered at a dose of about 1,540 μg/kg. In some aspects, an IL-7 protein is administered at a dose of about 1,560 μg/kg. In some aspects, an IL-7 protein is administered at a dose of about 1,580 μg/kg. In some aspects, an IL-7 protein is administered at a dose of about 1,600 μg/kg. In some aspects, an IL-7 protein is administered at a dose of about 1,620 μg/kg. In some aspects, an IL-7 protein is administered at a dose of about 1,640 μg/kg. In some aspects, an IL-7 protein is administered at a dose of about 1,660 μg/kg. In some aspects, an IL-7 protein is administered at a dose of about 1,680 μg/kg. In some aspects, an IL-7 protein is administered at a dose of about 1,700 μg/kg. In some aspects, an IL-7 protein is administered at a dose of about 1,720 μg/kg. In some aspects, an IL-7 protein is administered at a dose of about 1,740 μg/kg. In some aspects, an IL-7 protein is administered at a dose of about 1,760 μg/kg. In some aspects, an IL-7 protein is administered at a dose of about 1,780 μg/kg. In some aspects, an IL-7 protein is administered at a dose of about 1,800 μg/kg. In some aspects, an IL-7 protein is administered at a dose of about 1,820 μg/kg. In some aspects, an IL-7 protein is administered at a dose of about 1,840 μg/kg. In some aspects, an IL-7 protein is administered at a dose of about 1,860 μg/kg. In some aspects, an IL-7 protein is administered at a dose of about 1,880 μg/kg. In some aspects, an IL-7 protein is administered at a dose of about 1,900 μg/kg. In some aspects, an IL-7 protein is administered at a dose of about 1,920 μg/kg. In some aspects, an IL-7 protein is administered at a dose of about 1,940 μg/kg. In some aspects, an IL-7 protein is administered at a dose of about 1,960 μg/kg. In some aspects, an IL-7 protein is administered at a dose of about 1,980 μg/kg. In some aspects, an IL-7 protein is administered at a dose of about 2,000 μg/kg.

In some aspects, an IL-7 protein can be administered at a flat dose. In some aspects, an IL-7 protein can be administered at a flat dose of about 0.25 mg to about 9 mg. In some aspects, an IL-7 protein can be administered at a flat dose of about 0.25 mg, about 1 mg, about 3 mg, about 6 mg, or about 9 mg.

In some aspects, a subject disclosed herein receives a single administration of an IL-7 protein at any of the doses described above. In some aspects, an IL-7 protein disclosed herein is administered to a subject at multiple doses (i.e., repeated administrations). In some aspects, an IL-7 protein is administered to the subject at least two times, at least three times, at least four times, at least five times, at least six times, at least seven times, at least eight times, at least nine times, or at least ten times or more.

Where a subject (e.g., afflicted with a tumor) receives multiple administration of an IL-7 protein, in some aspects, the IL-7 protein is administered at a dosing frequency of about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks, e.g., at one of the doses described above. In some aspects, an IL-7 protein is administered at a dosing frequency of about once every 10 days, about once every 20 days, about once every 30 days, about once every 40 days, about once every 50 days, about once every 60 days, about once every 70 days, about once every 80 days, about once every 90 days, or about once every 100 days. In some aspects, the IL-7 protein is administered once in three weeks. In some aspects, the IL-7 protein is administered once a week. In some aspects, the IL-7 protein is administered once in two weeks. In some aspects, the IL-7 protein is administered once in four weeks. In some aspects, the IL-7 protein is administered once in six weeks. In some aspects, the IL-7 protein is administered once in eight weeks. In some aspects, the IL-7 protein is administered once in nine weeks. In some aspects, the IL-7 protein is administered once in 12 weeks. In some aspects, the IL-7 protein is administered once every 10 days. In some aspects, the IL-7 protein is administered once every 20 days. In some aspects, the IL-7 protein is administered once every 30 days. In some aspects, the IL-7 protein is administered once every 40 days. In some aspects, the IL-7 protein is administered once every 50 days. In some aspects, the IL-7 protein is administered once every 60 days. In some aspects, the IL-7 protein is administered once every 70 days. In some aspects, the IL-7 protein is administered once every 80 days. In some aspects, the IL-7 protein is administered once every 90 days. In some aspects, the IL-7 protein is administered once every 100 days. As demonstrated herein, in some aspects an IL-7 protein is administered at a dose of about 1,200 μg/kg and at a dosing interval of 8 about weeks.

In some aspects, a VEGF antagonist can be administered to a subject (e.g., in combination with an IL-7 protein described herein) at any suitable dose and/or dosing frequency to treat a tumor described herein. In some aspects, a VEGF antagonist is administered to a subject at a dose of about 0.001 mg/kg to about 20 mg/kg. In some aspects, a VEGF antagonist is administered at a dose of between about 1 mg/kg to about 20 mg/kg. In some aspects, a VEGF antagonist (e.g., bevacizumab) is administered at a dose of between about 5 mg/kg to about 15 mg/kg. In some aspects, a VEGF antagonist (e.g., bevacizumab) is administered at a dose of about 5 mg/kg, about 7.5 mg/kg, about 10 mg/kg, about 12.5 mg/kg, or about 15 mg/kg. In some aspects, a VEGF antagonist is administered to the subject at a dose of about 5 mg/kg. In some aspects, a VEGF antagonist is administered to the subject at a dose of about 7.5 mg/kg. In some aspects, a VEGF antagonist is administered to the subject at a dose of about 10 mg/kg. In some aspects, a VEGF antagonist is administered to the subject at a dose of about 12.5 mg/kg. In some aspects, a VEGF antagonist is administered to the subject at a dose of about 15 mg/kg. In some aspects, a VEGF antagonist (e.g., aflibercept) is administered to a subject at a dose of between about 4 mg/kg to about 15 mg/kg. In some aspects, a VEGF antagonist (e.g., aflibercept) is administered at a dose of about 4 mg/kg.

In some aspects, a subject disclosed herein receives a single administration of a VEGF antagonist (e.g., in combination with an IL-7 protein) at any of the doses described above. In some aspects, a VEGF antagonist disclosed herein is administered to a subject (e.g., alone or in combination with an IL-7 protein) at multiple doses (i.e., repeated administrations). In some aspects, a VEGF antagonist is administered to the subject at least two about times, at least about three times, at least about four times, at least about five times, at least about six times, at least about seven times, at least about eight times, at least about nine times, or at least about ten times or more.

Where a subject (e.g., afflicted with a tumor) receives multiple administration of a VEGF antagonist (e.g., alone or in combination with an IL-7 protein), in some aspects, the VEGF antagonist is administered at a dosing frequency of about once a day, about once every two days, about once every three days, about once every four days, about once every five days, about once every six days, about once a week, about once in two weeks, about once in three weeks, about once in four weeks, about once in five weeks, about once in six weeks, about once in seven weeks, about once in eight weeks, about once in nine weeks, about once in 10 weeks, about once in 11 weeks, or about once in 12 weeks, e.g., at one of the doses described herein. As demonstrated herein, in some aspects, a VEGF antagonist (e.g., bevacizumab) is administered at a dose of about 10 mg/kg and at a dosing interval of about 2 weeks.

Accordingly, in some aspects, a subject described herein (e.g., afflicted with a tumor) receives a single dose of an IL-7 protein (e.g., at one of the doses described herein) and a single dose of a VEGF antagonist (e.g., at one of the doses described herein). In such aspects, the IL-7 protein and the VEGF antagonist can be administered to the subject concurrently. For instance, in some aspects, an IL-7 protein and a VEGF antagonist can be administered concurrently as a single composition in a pharmaceutically acceptable carrier. In some aspects, an IL-7 protein and a VEGF antagonist can be administered concurrently as separate compositions. In some aspects, an IL-7 protein and a VEGF antagonist are administered to the subject sequentially. Where the IL-7 protein and the VEGF antagonist are administered to a subject sequentially, in some aspects, the duration of time between the administration of the IL-7 protein and the administration of the VEGF antagonist is at least about one day, at least about two days, at least about three days, at least about four days, at least about five days, at least about six days, at least about one week, at least about two weeks, at least about three weeks, at least about four weeks, at least about five weeks, at least about six weeks, at least about seven weeks, at least about eight weeks, at least about nine weeks, at least about ten weeks, at least about 11 weeks, at least about 12 weeks or more. In some aspects, the IL-7 protein is administered to the subject prior to the administration of the VEGF antagonist. In some aspects, the IL-7 protein is administered to the subject after the administration of the VEGF antagonist.

In some aspects, a subject described herein (e.g., afflicted with a tumor) receives a single dose of an IL-7 protein (e.g., at one of the doses described herein) and multiple doses (e.g., at least about two, at least about three, or at least about four) of a VEGF antagonist. In some aspects, a subject described herein (e.g., afflicted with a tumor) receives multiple doses of an IL-7 protein (e.g., at one of the doses described herein) and a single dose of a VEGF antagonist (e.g., at one of the doses described herein). In some aspects, a subject described herein (e.g., afflicted with a tumor) receives multiple doses of an IL-7 protein (e.g., at one of the doses described herein) and multiple doses a VEGF antagonist (e.g., at one of the doses described herein).

Where multiple doses of an IL-7 protein and/or a VEGF antagonist are administered to a subject, in some aspects, at least one of the doses of the IL-7 protein and at least one of the doses of the VEGF antagonist are administered to the subject concurrently (e.g., as a single composition or two separate compositions). In some aspects, each of the multiple doses of the IL-7 protein and each of the multiple doses of the VEGF antagonist are administered to the subject concurrently (e.g., as a single composition or two separate compositions). In some aspects, one or more of the multiple doses of the IL-7 protein and one or more of the multiple doses of the VEGF antagonist are administered to the subject sequentially. In some aspects, (i) at least one of the multiple doses of the IL-7 protein and at least one of the multiple doses of the VEGF antagonist are administered concurrently (e.g., as a single composition or two separate compositions), and (ii) at least one of the multiple doses of the IL-7 protein and at least one of the multiple doses of the VEGF antagonist are administered sequentially (e.g., on separate days).

As is apparent from the present disclosure, in some aspects, a subject receives multiple cycles of the treatment regimen described herein (IL-7 protein in combination with a VEGF antagonist). In some aspects, a subject receives at least about two, at least about three, at least about four, at least about five, at least about six, at least about seven, at least about eight, at least about nine, at least about 10 cycles of the treatment regimen. In some aspects, the subject receives two cycles of the treatment regimen. In some aspects, the subject receives three cycles of the treatment regimen. In some aspects, the subject receives four cycles of the treatment regimen. In some aspects, the subject receives five cycles of the treatment regimen. In some aspects, the subject receives six cycles of the treatment regimen. In some aspects, the subject receives seven cycles of the treatment regimen. In some aspects, the subject receives eight cycles of the treatment regimen. In some aspects, the subject receives nine cycles of the treatment regimen. In some aspects, the subject receives 10 cycles of the treatment regimen. Where multiple cycles of a treatment regimen are administered to a subject, in some aspects, each of the treatment cycles are the same. In some aspects, one or more of the treatment cycles are different (e.g., different dose and/or dosing interval).

In some aspects, each treatment cycle is about one week, about two weeks, about three weeks, about four weeks, about five weeks, about six weeks, about seven weeks, about eight weeks, about nine weeks, about 10 weeks, about 11 weeks, or about 12 weeks long. As demonstrated herein, in some aspects, each treatment cycle is about 8 weeks long. Unless indicated otherwise, each treatment cycle comprises at least one dose of an IL-7 protein and at least one dose of a VEGF antagonist. In some aspects, each treatment cycle comprises one dose of the IL-7 protein and one dose of the VEGF antagonist. In some aspects, each treatment cycle comprises one dose of the IL-7 protein and two doses of the VEGF antagonist. In some aspects, each treatment cycle comprises one dose of the IL-7 protein and three doses of the VEGF antagonist. In some aspects, each treatment cycle comprises one dose of the IL-7 protein and four doses of the VEGF antagonist. In some aspects, each treatment cycle comprises one dose of the IL-7 protein and five doses of the VEGF antagonist. As demonstrated herein (see, e.g., Example 3), in some aspects, each treatment cycle comprises one dose of an IL-7 protein and four doses of a VEGF antagonist, which are administered at a dosing interval of two weeks. For instance, in some aspects, the IL-7 protein and the VEGF antagonist are administered to the subject on day 1 of the treatment cycle, and then the additional doses of the VEGF antagonist area administered on days 15, 29, and 43 of the treatment cycle.

In some aspects, a combination treatment of an IL-7 protein and a VEGF antagonist described herein can be used with one or more additional therapeutic agents. Non-limiting examples of such additional therapeutic agents include: chemotherapy drugs, small molecule drugs, radiation, antibodies that stimulate the immune response to a given cancer, or a combination thereof. Accordingly, in some aspects, provided herein is a method of treating a tumor in a subject in need thereof, comprising administering to the subject an IL-7 protein in combination with a VEGF antagonist and an additional therapeutic agent. Non-limiting examples of such combinations can include: a therapy that enhances tumor antigen presentation (e.g., dendritic cell vaccine, GM-CSF secreting cellular vaccines, CpG oligonucleotides, imiquimod); a therapy that inhibits negative immune regulation e.g., by inhibiting CTLA-4 and/or PD1/PD-L1/PD-L2 pathway and/or depleting or blocking Tregs or other immune suppressing cells (e.g., myeloid-derived suppressor cells); a therapy that stimulates positive immune regulation, e.g., with agonists that stimulate the CD-137, OX-40, and/or CD40 or GITR pathway and/or stimulate T cell effector function; a therapy that increases systemically the frequency of anti-tumor T cells; a therapy that depletes or inhibits Tregs, such as Tregs in the tumor, e.g., using an antagonist of CD25 (e.g., daclizumab) or by ex vivo anti-CD25 bead depletion; a therapy that impacts the function of suppressor myeloid cells in the tumor; a therapy that enhances immunogenicity of tumor cells (e.g., anthracyclines); adoptive T cell or NK cell transfer including genetically modified cells, e.g., cells modified by chimeric antigen receptors (CAR-T therapy); a therapy that inhibits a metabolic enzyme such as indoleamine dioxigenase (IDO), dioxigenase, arginase, or nitric oxide synthetase; a therapy that reverses/prevents T cell anergy or exhaustion; a therapy that triggers an innate immune activation and/or inflammation at a tumor site; administration of immune stimulatory cytokines; or blocking of immuno repressive cytokines.

In some aspects, an additional therapeutic agent that can be used with the combination treatment of an IL-7 protein and a VEGF antagonist comprises an immune checkpoint inhibitor (i.e., blocks signaling through the particular immune checkpoint pathway). Accordingly, in some aspects, present disclosure provides a method of treating a tumor in a subject in need thereof, comprising administering to the subject an IL-7 protein in combination with both a VEGF antagonist and an immune checkpoint inhibitor. Non-limiting examples of immune checkpoint inhibitors that can be used in the present methods comprise a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or combinations thereof. Non-limiting examples of such immune checkpoint inhibitors are known in the art. For example, suitable anti-PD-1 antagonists are described, e.g., in U.S. Pat. Nos. 6,808,710; 7,488,802; 8,008,449; 8,168,757; 8,354,509; 8,609,089; 8,779,105; and 9,217,034; and 7,635,757; 7,943,743; 8,217,149; 8,981,063; and 9,624,298, each of which is incorporated herein by reference in its entirety. Suitable anti-CTLA-4 antagonists are described, e.g., in U.S. Pat. Nos. 5,977,318; 6,051,227; 6,682,736; 6,984,720; 7,034,121; 7,605,238; 10,479,833; and International Publ. No. WO 2007/113648, each of which is incorporated herein by reference in its entirety. Suitable TIM-3 antagonists are described, e.g., in U.S. Pat. Nos. 10,508,149; 10,533,052; 10,894,830, each of which is incorporated hereby reference in its entirety.

In some aspects, an additional therapeutic agent that can be used comprises an immune checkpoint activator (i.e., promotes signaling through the particular immune checkpoint pathway). In some aspects, immune checkpoint activator comprises OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or any combination thereof.

In some aspects, a treatment regimen provided herein (an IL-7 protein in combination with a VEGF antagonist) is not used in combination with one or more other anti-cancer treatments. Accordingly, in some aspects, a subject that is being treated with an IL-7 protein in combination with a VEGF antagonist does not receive one or more of the following anti-cancer treatments: (1) any concomitant treatment, including chemotherapy, hormone therapy, immunotherapy, radiotherapy, investigational drug or herbal therapy, approved or experimental for the treatment of cancer; (2) immunosuppressants, including but not limited to cyclophosphamide, azathioprine, methotrexate. and thalidomide; (3) granulocyte colony-stimulating factor and granulocyte macrophage colony-stimulating factor (e.g., pegfilgrastim); and (4) immune stimulants (e.g., IFN-α, IFN-7, and IL-2).

### II.A. IL-7 Proteins Useful for the Disclosure

Disclosed herein are IL-7 proteins that can be used in combination with a VEGF antagonist to treat a cancer (or a tumor). In some aspects, IL-7 protein useful for the present uses can be wild-type IL-7 or modified IL-7 (i.e., not wild-type IL-7 protein) (e.g., IL-7 variant, IL-7 functional fragment, IL-7 derivative, or any combination thereof, e.g., fusion protein, chimeric protein, etc.) as long as the IL-7 protein contains one or more biological activities of IL-7, e.g., capable of binding to IL-7R, e.g., inducing early T-cell development, promoting T-cell homeostasis. See ElKassar and Gress. J Immunotoxicol. 2010 March; 7(1): 1-7. In some aspects, an IL-7 protein of the present disclosure is not a wild-type IL-7 protein (i.e., comprises one or more modifications). Non-limiting examples of such modifications can include an oligopeptide and/or a half-life extending moiety. See WO 2016/200219, which is herein incorporated by reference in its entirety.

IL-7 binds to its receptor which is composed of the two chains IL-7Ra (CD127), shared with the thymic stromal lymphopoietin (TSLP) (Ziegler and Liu, 2006), and the common γ chain (CD132) for IL-2, IL-15, IL-9 and IL-21. Whereas γc is expressed by most hematopoietic cells, IL-7Rα is nearly exclusively expressed on lymphoid cells. After binding to its receptor, IL-7 signals through two different pathways: Jak-Stat (Janus kinase-Signal transducer and activator of transcription) and PI3K/Akt responsible for differentiation and survival, respectively. The absence of IL-7 signaling is responsible for a reduced thymic cellularity as observed in mice that have received an anti-IL-7 neutralizing monoclonal antibody (MAb); Grabstein et al., 1993), in IL-7−/− (von Freeden-Jeffry et al., 1995), IL-7Rα−/− (Peschon et al., 1994; Maki et al., 1996), γc−/−(Malissen et al., 1997), and Jak3−/− mice (Park et al., 1995). In the absence of IL-7 signaling, mice lack T-, B-, and NK-T cells. IL-7α−/−mice (Peschon et al., 1994) have a similar but more severe phenotype than IL-7−/− mice (von Freeden-Jeffry et al., 1995), possibly because TSLP signaling is also abrogated in IL-7α−/− mice. IL-7 is required for the development of 76 cells (Maki et al., 1996) and NK-T cells (Boesteanu et al., 1997).

In some aspects, an IL-7 protein useful for the present disclosure comprises an amino acid sequence as set forth in any one of SEQ ID NOs: 1 to 6. In some aspects, the IL-7 protein comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, to a sequence of SEQ ID NOS: 1 to 6. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 1. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 2. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 3. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 4. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 5. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 6.

In some aspects, the IL-7 protein includes a modified IL-7 or a fragment thereof, wherein the modified IL-7 or the fragment retains one or more biological activities of wild-type IL-7. Accordingly, in some aspects, an IL-7 protein useful for the present disclosure comprises a fragment of the amino acid sequence set forth in any one of SEQ ID NOs: 1-6, wherein the fragment retains one or more biological activities of the wild-type IL-7 protein. In some aspects, the IL-7 protein can be derived from humans, rats, mice, monkeys, cows, or sheep.

In some aspects, the human IL-7 can have an amino acid sequence represented by SEQ ID NO: 1 (Genbank Accession No. P13232); the rat IL-7 can have an amino acid sequence represented by SEQ ID NO: 2 (Genbank Accession No. P56478); the mouse IL-7 can have an amino acid sequence represented by SEQ ID NO: 3 (Genbank Accession No. P10168); the monkey IL-7 can have an amino acid sequence represented by SEQ ID NO: 4 (Genbank Accession No. NP 001279008); the cow IL-7 can have an amino acid sequence represented by SEQ ID NO: 5 (Genbank Accession No. P26895), and the sheep IL-7 can have an amino acid sequence represented by SEQ ID NO: 6 (Genbank Accession No. Q28540). As will be apparent to those skilled in the art, an IL-7 protein provided in any of SEQ ID NOs: 1-6 has a single peptide, but the mature form of an IL-7 protein would not comprise the signal peptide. Accordingly, as is apparent from the present disclosure, in some aspects, an IL-7 protein that can be used with the methods provided herein lack a signal peptide. Amino acid sequences for such exemplary TL-7 proteins are provided in SEQ ID NOs: 80 to 85 (see also Table 1A).

Accordingly, in some aspects, an IL-7 protein useful for the present disclosure comprises an amino acid sequence having a sequence identity of about 70%, about 75%, about 80%, about 85%, about 90%, about 91%, about 92%, about 93%, about 94%, about 95%, about 96%, about 97%, about 98%, or about 99% or higher, as compared to the amino acid sequence set forth in any one of SEQ ID NOs: 80 to 85. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 80. In some aspects, the IL-7 protein comprises the amino acid sequence set forth in SEQ ID NO: 80. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 81. In some aspects, the IL-7 protein comprises the amino acid sequence set forth in SEQ ID NO: 81. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 82. In some aspects, the IL-7 protein comprises the amino acid sequence set forth in SEQ ID NO: 82. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 83. In some aspects, the IL-7 protein comprises the amino acid sequence set forth in SEQ ID NO. 83. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 84. In some aspects, the IL-7 protein comprises the amino acid sequence set forth in SEQ ID NO: 84. In some aspects, the IL-7 protein comprises an amino acid sequence having at least about 70%, about 75%, about 80%, about 85%, about 90%, about 91%, about 92%, about 93%, about 94%, about 95%, about 96%, about 97%, about 98%, or about 99% to the sequence set forth in SEQ ID NO: 85. In some aspects, the IL-7 protein comprises the amino acid sequence set forth in SEQ ID NO: 85.

In some aspects, an IL-7 protein useful for the present disclosure comprises an IL-7 fusion protein. In some aspects, an IL-7 fusion protein comprises (i) an oligopeptide and (i) an IL-7 or a variant thereof (e.g., IL-7 protein set forth in any one of SEQ ID NOs: 1-6 and 80-85). In some aspects, the oligopeptide is linked to the N-terminal region of the IL-7 or a variant thereof.

In some aspects, an oligopeptide disclosed herein consists of 1 to 10 amino acids. In some aspects, an oligopeptide consists of at least 1, at least 2, at least 3, at least 4, at least 5, at least 6, at least 7, at least 8, at least 9, or 10 amino acids. In some aspects, one or more amino acids of an oligopeptide are selected from the group consisting of methionine, glycine, and combinations thereof. In some aspects, an oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. In some aspects, an oligopeptide is methionine-glycine-methionine.

In some aspects, an IL-7 fusion protein comprises (i) an IL-7 or a variant thereof, and (ii) a half-life extending moiety. In some aspects, a half-life extending moiety extends the half-life of the IL-7 or variant thereof. In some aspects, a half-life extending moiety is linked to the C-terminal region of an IL-7 or a variant thereof.

In some aspects, an IL-7 fusion protein comprises (i) IL-7 (a first domain), (ii) a second domain that includes an amino acid sequence having 1 to 10 amino acid residues consisting of methionine, glycine, or a combination thereof, e.g., MGM, and (iii) a third domain comprising a half-life extending moiety. In some aspects, the half-life extending moiety can be linked to the N-terminal or the C-terminal of the first domain or the second domain. Additionally, the IL-7 including the first domain and the second domain can be linked to both terminals of the third domain.

Non-limiting examples of half-life extending moieties include: Fc, albumin, an albumin-binding polypeptide, Pro/Ala/Ser (PAS), a C-terminal peptide (CTP) of the 3 subunit of human chorionic gonadotropin, polyethylene glycol (PEG), long unstructured hydrophilic sequences of amino acids (XTEN), hydroxyethyl starch (HES), an albumin-binding small molecule, and combinations thereof.

In some aspects, a half-life extending moiety is Fc. In some aspects, Fc is from a modified immunoglobulin in which the antibody-dependent cellular cytotoxicity (ADCC) or complement-dependent cytotoxicity (CDC) weakened due to the modification in the binding affinity with the Fc receptor and/or a complement. In some aspects, the modified immunoglobulin can be selected from the group consisting of IgG1, IgG2, IgG3, IgG4, IgA1, IgA2, IgD, IgE, and a combination thereof. In some aspects, an Fc is a hybrid Fc (“hFc” or “hyFc”), comprising a hinge region, a CH2 domain, and a CH3 domain. In some aspects, a hinge region of a hybrid Fc disclosed herein comprises a human IgD hinge region. In some aspects, a CH2 domain of a hybrid Fc comprises a part of human IgD CH2 domain and a part of human IgG4 CH2 domain. In some aspects, a CH3 domain of a hybrid Fc comprises a part of human IgG4 CH3 domain. Accordingly, in some aspects, a hybrid Fc disclosed herein comprises a hinge region, a CH2 domain, and a CH3 domain, wherein the hinge region comprises a human IgD hinge region, wherein the CH2 domain comprises a part of human IgD CH2 domain and a part of human IgG4 CH2 domain, and wherein the CH3 domain comprises a part of human IgG4 CH3 domain.

In some aspects, an Fe disclosed herein can be an Fc variant. As used herein, the term “Fc variant” refers to an Fc which was prepared by substituting a part of the amino acids among the Fc region or by combining the Fc regions of different kinds. The Fc region variant can prevent from being cut off at the hinge region. Specifically, in some aspects, a Fc variant comprises modifications at the 144^{th} amino acid and/or 145^{th} amino acid of SEQ ID NO: 9. In some aspects, the 144^{th} amino acid (K) and/or the 145^{th} amino acid (K) is substituted with G or S.

In some aspects, an Fc or an Fc variant disclosed herein can be represented by the following formula: N′—(Z1)p-Y-Z2-Z3-Z4-C, wherein:
N′ comprises the N-terminus;
Z1 comprises an amino acid sequence having 5 to 9 consecutive amino acid residues from the amino acid residue at position 98 toward the N-terminal, among the amino acid residues at positions from 90 to 98 of SEQ ID NO: 7;
Y comprises an amino acid sequence having 5 to 64 consecutive amino acid residues from the amino acid residue at position 162 toward the N-terminal, among the amino acid residues at positions from 99 to 162 of SEQ ID NO: 7;
Z2 comprises an amino acid sequence having 4 to 37 consecutive amino acid residues from the amino acid residue at position 163 toward the C-terminal, among the amino acid residues at positions from 163 to 199 of SEQ ID NO: 7;
Z3 comprises an amino acid sequence having 71 to 106 consecutive amino acid residues from the amino acid residue at position 220 toward the N-terminal, among the amino acid residues at positions from 115 to 220 of SEQ ID NO: 8; and
Z4 comprises an amino acid sequence having 80 to 107 consecutive amino acid residues from the amino acid residue at position 221 toward the C-terminal, among the amino acid residues at positions from 221 to 327 of SEQ ID NO: 8.

In some aspects, a Fc region disclosed herein can include the amino acid sequence of SEQ ID NO: 9 (hyFc), SEQ ID NO: 10 (hyFcM1), SEQ ID NO: 11 (hyFcM2), SEQ ID NO: 12 (hyFcM3), or SEQ ID NO: 13 (hyFcM4). In some aspects, the Fc region can include the amino acid sequence of SEQ ID NO: 14 (a non-lytic mouse Fc).

Other non-limiting examples of Fc regions that can be used with the present disclosure are described in U.S. Pat. No. 7,867,491, which is herein incorporated by reference in its entirety.

In some aspects, an IL-7 fusion protein disclosed herein comprises both an oligopeptide and a half-life extending moiety.

In some aspects, an IL-7 protein can be fused to albumin, a variant, or a fragment thereof. Examples of the IL-7-albumin fusion protein can be found at International Application Publication No. WO 2011/124718 A1. In some aspects, an IL-7 protein is fused to a pre-pro-B cell Growth Stimulating Factor (PPBSF), optionally by a flexible linker. See US 2002/0058791A1. In some aspects, an IL-7 protein useful for the disclosure is an IL-7 conformer that has a particular three dimensional structure. See US 2005/0249701 A1. In some aspects, an IL-7 protein can be fused to an Ig chain, wherein amino acid residues 70 and 91 in the IL-7 protein are glycosylated the amino acid residue 116 in the IL-7 protein is non-glycosylated. See U.S. Pat. No. 7,323,549 B2. In some aspects, an IL-7 protein that does not contain potential T-cell epitopes (thereby to reduce anti-IL-7 T-cell responses) can also be used for the present disclosure. See US 2006/0141581 A1. In some aspects, an IL-7 protein that has one or more amino acid residue mutations in carboxy-terminal helix D region can be used for the present disclosure. The IL-7 mutant can act as IL-7R partial agonist despite lower binding affinity for the receptor. See US 2005/0054054A1. Any IL-7 proteins described in the above listed patents or publications are incorporated herein by reference in their entireties.

In addition, non-limiting examples of additional IL-7 proteins useful for the present disclosure are described in U.S. Pat. Nos. 7,708,985, 8,034,327, 8,153,114, 7,589,179, 7,323,549, 7,960,514, 8,338,575, 7,118,754, 7,488,482, 7,670,607, 6,730,512, WO0017362, GB2434578A, WO 2010/020766 A2, WO91/01143, Beq et al., Blood, vol. 114 (4), 816, 23 Jul. 2009, Kang et al., J. Virol. Doi:10.1128/JVI.02768-15, Martin et al., Blood, vol. 121 (22), 4484, May 30, 2013, McBride et al., Acta Oncologica, 34:3, 447-451, Jul. 8, 2009, and Xu et al., Cancer Science, 109: 279-288, 2018, which are incorporated herein by reference in their entireties.

In some aspects, an oligopeptide disclosed herein is directly linked to the N-terminal region of IL-7 or a variant thereof. In some aspects, an oligopeptide is linked to the N-terminal region via a linker. In some aspects, a half-life extending moiety disclosed herein is directly linked to the C-terminal region of IL-7 or a variant thereof. In some aspects, a half-life extending moiety is linked to the C-terminal region via a linker. In some aspects, a linker is a peptide linker. In some aspects, a peptide linker comprises a peptide of 10 to 20 amino acid residues consisting of Gly and Ser residues. In some aspects, a linker is an albumin linker. In some aspects, a linker is a chemical bond. In some aspects, a chemical bond comprises a disulfide bond, a diamine bond, a sulfide-amine bond, a carboxy-amine bond, an ester bond, a covalent bond, or combinations thereof. When the linker is a peptide linker, in some aspects, the connection can occur in any linking region. They can be coupled using a crosslinking agent known in the art. In some aspects, examples of the crosslinking agent can include N-hydroxy succinimide esters such as 1,1-bis(diazoacetyl)-2-phenylethane, glutaraldehyde, and 4-azidosalicylic acid; imido esters including disuccinimidyl esters such as 3,3′-dithiobis (succinimidyl propionate), and bifunctional maleimides such as bis-Nmaleimido-1,8-octane, but is not limited thereto.

In some aspects, an IL-7 (or variant thereof) portion of IL-7 fusion protein disclosed herein comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 15-20. In some aspects, an IL-7 (or variant thereof) portion of IL-7 fusion protein disclosed herein comprises the amino acid sequence set forth in SEQ ID NOs: 15-20.

In some aspects, an IL-7 fusion protein comprises: a first domain including a polypeptide having the activity of IL-7 or a similar activity thereof; a second domain comprising an amino acid sequence having 1 to 10 amino acid residues consisting of methionine, glycine, or a combination thereof, and a third domain, which is an Fc region of modified immunoglobulin, coupled to the C-terminal of the first domain.

In some aspects, an IL-7 fusion protein that can be used with the present methods comprises an amino sequence that is at least 70%, at least 75%, at least 80%, at least 85%, at least 90%, at least 91%, at least 92%, at least 93%, at least 94%, at least 95%, at least 96%, at least 97%, at least 98%, at least 98%, or at least 99% identical to an amino acid sequence set forth in SEQ ID NOs: 21-25. In some aspects, an IL-7 fusion protein of the present disclosure comprises the amino acid sequence set forth in SEQ ID NOs: 21-25. In some aspects, an IL-7 fusion protein disclosed herein comprises the amino acid sequence set forth in SEQ ID NOs: 26 and 27.

Unless indicated otherwise, an IL-7 protein described (including fusion proteins) herein can be administered to a subject (e.g., in combination with a VEGF antagonist) using any suitable routes of administration. In some aspects, the IL-7 protein is administered to the subject intravenously, parenthetically, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally. In some aspects, the IL-7 protein is administered intramuscularly to the subject.

### II.B. VEGF Antagonists

In some aspects, the present disclosure provides a method of treating a tumor in a subject in need thereof, comprising administering to the subject an IL-7 protein in combination with a VEGF antagonist. As used herein, the term “VEGF antagonist” refers to any molecule that is capable of neutralizing, blocking, inhibiting, abrogating, reducing, and/or interfering with VEGF activities, including its binding to one or more VEGF receptors. VEGF antagonists include molecules which interfere with the interaction between VEGF and a natural VEGF receptor, e.g., molecules which bind to VEGF or a VEGF receptor and prevent or otherwise hinder the interaction between VEGF and a VEGF receptor. Accordingly, in some aspects, a VEGF antagonist described herein can specifically target VEGF itself. In some aspects, a VEGF antagonist described herein specifically targets one or more receptors of VEGF.

Unless indicated otherwise, a VEGF antagonist useful for the present disclosure can be a protein, peptide, nucleic acid, small molecule, or a combination thereof. In some aspects, a VEGF antagonist useful for the present disclosure comprises an antisense-oligonucleotide, siRNA, shRNA, miRNA, dsRNA, aptamer, PNA (peptide nucleic acid) targeting VEGF, or a vector including the same. In some aspects, a VEGF antagonist comprises an antibody, or an antigen-binding fragment thereof, that specifically binds to the VEGF or one or more of the VEGF receptor (VEGFR) (“anti-VEGF antibody” and “anti-VEGFR antibody,” respectively), a polynucleotide encoding the anti-VEGF antibody and/or anti-VEGFR antibody, or a vector comprising the polynucleotide thereof.

Non-limiting examples of suitable VEGF antagonists are known in the art and include, e.g., comprises aflibercept (EYLEA® and ZALTRAP®), bevacizumab (AVASTIN®, ZIRABEV®, and MVASI®), ranibizumab (LUCENTIS®, BYOOVIZ®, SUSVIMO®), ramucirumab (CYRAMZA®), sorafenib (NEXAVAR®), sunitinib (SUTENT®), pazopanib (VOTRIENT), dasatinib (SPRYCEL®), regorafenib (STIVARGA®), cabozantinib (CABOMETYX®, COMETRIQ®), lenvatinib (LENVIMA®), ponatinib (ICLUSIG®), axitinib (INLYTA®), tivozanib (FOTIVDA®), vandetanib (CAPRELSA®), or a combination thereof.

In some aspects, a VEGF antagonist useful for the present disclosure is an anti-VEGF antibody. In some aspects, the VEGF antagonist is bevacizumab, e.g., as described in U.S. Publ. No. 2015/0147317, which is incorporated herein by reference in its entirety. In some aspects, the VEGF antagonist is a variant of bevacizumab. For instance, in some aspects, such a variant cross-competes with bevacizumab. In some aspects, the bevacizumab variant binds to the same epitope as bevacizumab. In some aspects, the bevacizumab variant has the same CDRs as bevacizumab. Accordingly, in some aspects, a method provided herein (e.g., to treat tumor) comprises administering to a subject in need thereof an IL-7 protein in combination with bevacizumab (or a variant thereof). For such aspects, bevacizumab (or a variant thereof) can be administered to the subject at any suitable doses known in the art (e.g., FDA approved doses), such as between about 5 mg/kg to about 15 mg/kg. For example, in some aspects, bevacizumab (or a variant thereof) is administered in combination with an IL-7 protein at a dose of about 5 mg/kg, about 7.5 mg/kg, about 10 mg/kg, about 12.5 mg/kg, or about 15 mg/kg. In some aspects, bevacizumab (or a variant thereof) is administered in combination with an IL-7 protein at a dose of about 10 mg/kg. As described herein, in some aspects, such a method further comprises administering an additional therapeutic agent to the subject (e.g., an immune checkpoint inhibitor).

In some aspects, a VEGF antagonist useful for the present disclosure is aflibercept, e.g., as described in U.S. Pat. No. 11,135,266, which is incorporated herein by reference in its entirety. In some aspects, the VEGF antagonist is a variant of aflibercept. In some aspects, the aflibercept variant cross-competes with aflibercept. In some aspects, the aflibercept binds to the same epitope as aflibercept. In some aspects, the aflibercept variant has the same CDRs as aflibercept. Accordingly, in some aspects, a method provided herein (e.g., to treat tumor) comprises administering to a subject in need thereof an IL-7 protein in combination with aflibercept (or a variant thereof). For such aspects, aflibercept (or a variant thereof) can be administered to the subject at any suitable doses known in the art (e.g., FDA approved doses), such as between about 4 mg/kg to about 15 mg/kg. For example, in some aspects, aflibercept (or a variant thereof) is administered in combination with an IL-7 protein at a dose of about 4 mg/kg. As described herein, in some aspects, such a method can further comprise administering an additional therapeutic agent to the subject (e.g., an immune checkpoint inhibitor).

In some aspects, a VEGF antagonist that can be used with the present disclosure is ranibizumab, e.g., as described in U.S. Pat. No. 7,169,901, which is incorporated herein by reference in its entirety. In some aspects, the VEGF antagonist is a variant of ranibizumab. In some aspects, the ranibizumab variant cross-competes with ranibizumab. In some aspects, the ranibizumab variant binds to the same epitope as ranibizumab. In some aspects, the ranibizumab variant has the same CDRs as ranibizumab. Accordingly, in some aspects, a method provided herein (e.g., to treat tumor) comprises administering to a subject in need thereof an IL-7 protein in combination with ranibizumab (or a variant thereof). For such aspects, ranibizumab (or a variant thereof) can be administered to the subject at any suitable doses known in the art (e.g., FDA approved doses). In some aspects, such a method further comprises administering an additional therapeutic agent to the subject (e.g., an immune checkpoint inhibitor).

In some aspects, a VEGF antagonist is ramucirumab, e.g., as described in U.S. Pat. No. 8,057,791, which is incorporated herein by reference in its entirety. In some aspects, the VEGF antagonist is a variant of ramucirumab. In some aspects, the ramucirumab variant cross-competes with ramucirumab. In some aspects, the ramucirumab variant binds to the same epitope as ramucirumab. In some aspects, the ramucirumab has the same CDRs as ramucirumab. Accordingly, in some aspects, a method provided herein (e.g., to treat tumor) comprises administering to a subject in need thereof an IL-7 protein in combination with ramucirumab (or a variant thereof). For such aspects, ramucirumab (or a variant thereof) can be administered to the subject at any suitable doses known in the art (e.g., FDA approved doses). In some aspects, such a method further comprises administering an additional therapeutic agent to the subject (e.g., an immune checkpoint inhibitor).

Unless indicated otherwise, a VEGF antagonist described herein can be administered to a subject (e.g., in combination with an IL-7 protein) using any suitable routes of administration. In some aspects, the VEGF antagonist is administered to the subject intravenously, parenthetically, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally. In some aspects, the VEGF antagonist is administered intravenously to the subject. Accordingly, in some aspects, a treatment method provided herein comprises administering to a subject at least one dose an IL-7 protein intramuscularly and at least one dose of a VEGF antagonist intravenously.

### III. Nucleic Acids, Vectors, Host Cells

Some aspects described herein pertain to one or more nucleic acid molecules that encode a therapeutic agent described herein (e.g., an IL-7 protein and/or a VEGF antagonist). The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and can or cannot contain intronic sequences. In some aspects, the nucleic acid is a cDNA molecule. Nucleic acids described herein can be obtained using standard molecular biology techniques known in the art.

Some nucleic acid molecules disclosed herein are those encoding an IL-7 protein (e.g., disclosed herein) that can be used in combination with a VEGF antagonist, e.g., to treat a tumor. Exemplary nucleic acid sequences encoding an IL-7 protein disclosed herein are set forth in SEQ ID NOs: 29-39.

In some aspects, the present disclosure provides a vector comprising an isolated nucleic acid molecule encoding a therapeutic agent disclosed herein (e.g., an IL-7 protein and/or a VEGF antagonist). In some aspects, a vector can be used for gene therapy.

When used as a gene therapy (e.g., in humans), a nucleic acid encoding a therapeutic agent disclosed herein (e.g., an IL-7 protein and/or a VEGF antagonist) can be administered at a dosage in the range of 0.1 mg to 200 mg. In some aspects, the dosage is in the range of 0.6 mg to 100 mg. In some aspects, the dosage is in the range of 1.2 mg to 50 mg.

Suitable vectors for the disclosure include expression vectors, viral vectors, and plasmid vectors. In some aspects, the vector is a viral vector.

As used herein, an expression vector refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

As used herein, viral vectors include, but are not limited to, nucleic acid sequences from the following viruses: retrovirus, such as Moloney murine leukemia virus, Harvey murine sarcoma virus, murine mammary tumor virus, and Rous sarcoma virus; lentivirus; adenovirus; adeno-associated virus; SV40-type viruses; polyomaviruses; Epstein-Barr viruses; papilloma viruses; herpes virus; vaccinia virus; polio virus; and RNA virus such as a retrovirus. One can readily employ other vectors well-known in the art. Some viral vectors are based on non-cytopathic eukaryotic viruses in which non-essential genes have been replaced with the gene of interest. Non-cytopathic viruses include retroviruses, the life cycle of which involves reverse transcription of genomic viral RNA into DNA with subsequent proviral integration into host cellular DNA.

In some aspects, a vector is derived from an adeno-associated virus. In some aspects, a vector is derived from a lentivirus. Examples of the lentiviral vectors are disclosed in WO9931251, WO9712622, WO9817815, WO9817816, and WO9818934, each which is incorporated herein by reference in its entirety.

Other vectors include plasmid vectors. Plasmid vectors have been extensively described in the art and are well-known to those of skill in the art. See, e.g., Sambrook et al., Molecular Cloning: A Laboratory Manual, Second Edition, Cold Spring Harbor Laboratory Press, 1989. In the last few years, plasmid vectors have been found to be particularly advantageous for delivering genes to cells in vivo because of their inability to replicate within and integrate into a host genome. These plasmids, however, having a promoter compatible with the host cell, can express a peptide from a gene operably encoded within the plasmid. Some commonly used plasmids available from commercial suppliers include pBR322, pUC18, pUC19, various pcDNA plasmids, pRC/CMV, various pCMV plasmids, pSV40, and pBlueScript. Additional examples of specific plasmids include pcDNA3.1, catalog number V79020; pcDNA3.1/hygro, catalog number V87020; pcDNA4/myc-His, catalog number V86320; and pBudCE4.1, catalog number V53220, all from Invitrogen (Carlsbad, CA.). Other plasmids are well-known to those of ordinary skill in the art. Additionally, plasmids can be custom designed using standard molecular biology techniques to remove and/or add specific fragments of DNA.

Also encompassed by the present disclosure is a method for making a therapeutic agent disclosed herein (e.g., an IL-7 protein). In some aspects, such a method can comprise expressing the therapeutic agent (e.g., an IL-7 protein) in a cell comprising a nucleic acid molecule encoding the therapeutic agent, e.g., SEQ ID NOs: 29-39. Additional details regarding the method for making an IL-7 protein disclosed herein are provided, e.g., in U.S. Pat. No. 11,041,007, which is herein incorporated by reference in its entirety. Host cells comprising these nucleotide sequences are encompassed herein. Non-limiting examples of host cell that can be used include immortal hybridoma cell, NS/0 myeloma cell, 293 cell, Chinese hamster ovary (CHO) cell, HeLa cell, human amniotic fluid-derived cell (CapT cell), COS cell, or combinations thereof.

### IV. Pharmaceutical Compositions

Further provided herein are compositions comprising one or more therapeutic agents (e.g., an IL-7 protein and/or a VEGF antagonist) having the desired degree of purity in a physiologically acceptable carrier, excipient or stabilizer (Remington's Pharmaceutical Sciences (1990) Mack Publishing Co., Easton, PA). In some aspects, a composition disclosed herein comprises an IL-7 protein or a VEGF antagonist. As disclosed herein, such compositions can be used in combination (e.g., a first composition comprising an IL-7 protein and a second composition comprising a VEGF antagonist). In some aspects, a composition disclosed herein comprises both an IL-7 protein and a VEGF antagonist.

Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, a composition disclosed herein comprises one or more additional components selected from: a bulking agent, stabilizing agent, surfactant, buffering agent, or combinations thereof.

Buffering agents useful for the current disclosure can be a weak acid or base used to maintain the acidity (pH) of a solution near a chosen value after the addition of another acid or base. Suitable buffering agents can maximize the stability of the pharmaceutical compositions by maintaining pH control of the composition. Suitable buffering agents can also ensure physiological compatibility or optimize solubility. Rheology, viscosity and other properties can also dependent on the pH of the composition. Common buffering agents include, but are not limited to, a Tris buffer, a Tris-Cl buffer, a histidine buffer, a TAE buffer, a HEPES buffer, a TBE buffer, a sodium phosphate buffer, a MES buffer, an ammonium sulfate buffer, a potassium phosphate buffer, a potassium thiocyanate buffer, a succinate buffer, a tartrate buffer, a DIPSO buffer, a HEPPSO buffer, a POPSO buffer, a PIPES buffer, a PBS buffer, a MOPS buffer, an acetate buffer, a phosphate buffer, a cacodylate buffer, a glycine buffer, a sulfate buffer, an imidazole buffer, a guanidine hydrochloride buffer, a phosphate-citrate buffer, a borate buffer, a malonate buffer, a 3-picoline buffer, a 2-picoline buffer, a 4-picoline buffer, a 3,5-lutidine buffer, a 3,4-lutidine buffer, a 2,4-lutidine buffer, a Aces, a diethylmalonate buffer, a N-methylimidazole buffer, a 1,2-dimethylimidazole buffer, a TAPS buffer, a bis-Tris buffer, a L-arginine buffer, a lactate buffer, a glycolate buffer, or combinations thereof.

In some aspects, a composition disclosed herein further comprises a bulking agent. Bulking agents can be added to a pharmaceutical product in order to add volume and mass to the product, thereby facilitating precise metering and handling thereof. Bulking agents that can be used with the present disclosure include, but are not limited to, sodium chloride (NaCl), mannitol, glycine, alanine, or combinations thereof.

In some aspects, a composition disclosed herein can also comprise a stabilizing agent. Non-limiting examples of stabilizing agents that can be used with the present disclosure include: sucrose, trehalose, raffinose, arginine, or combinations thereof.

In some aspects, a composition disclosed herein comprises a surfactant. In some aspects, the surfactant can be selected from the following: alkyl ethoxylate, nonylphenol ethoxylate, amine ethoxylate, polyethylene oxide, polypropylene oxide, fatty alcohols such as cetyl alcohol or oleyl alcohol, cocamide MEA, cocamide DEA, polysorbates, dodecyl dimethylamine oxide, or combinations thereof. In some aspects, the surfactant is polysorbate 20 or polysorbate 80.

In some aspects, a composition comprising an IL-7 protein can be formulated using the same formulation of a VEGF antagonist (e.g., which is to be used in combination with the IL-7 protein). In some aspects, an IL-7 protein and a VEGF antagonist are formulated using different formulations.

A pharmaceutical composition can be formulated for any route of administration to a subject. Specific examples of routes of administration include intramuscularly, subcutaneously, ophthalmic, intravenously, intraperitoneally, intradermally, intraorbitally, intracerebrally, intracranially, intraspinally, intraventricular, intrathecally, intracistemally, intracapsularly, or intratumorally. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN© 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An antibody or antigen-binding portion thereof described herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in one aspect, have diameters of less than 50 microns, in one aspect less than 10 microns.

A therapeutic agent disclosed herein (e.g., an IL-7 protein and/or a VEGF antagonist) can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In some aspects, a pharmaceutical composition comprising a therapeutic agent described herein (e.g., an IL-7 protein and/or a VEGF antagonist) is a lyophilized powder, which can be reconstituted for administration as solutions, emulsions and other mixtures. It can also be reconstituted and formulated as solids or gels. The lyophilized powder is prepared by dissolving an antibody or antigen-binding portion thereof described herein, or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. The solvent can also contain a buffer, such as citrate, sodium or potassium phosphate or other such buffer known to those of skill in the art at, in one aspect, about neutral pH. Subsequent sterile filtration of the solution followed by lyophilization under standard conditions known to those of skill in the art provides the desired formulation. In some aspects, the resulting solution can be apportioned into vials for lyophilization. Each vial can contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

The compositions to be used for in vivo administration can be sterile. This is readily accomplished by filtration through, e.g., sterile filtration membranes.

The following examples are merely illustrative and should not be construed as limiting the scope of this disclosure in any way as many variations and equivalents will become apparent to those skilled in the art upon reading the present disclosure.

### EXAMPLES

### Example 1: Effect of IL-7 Protein and VEGF Antagonist Combination Treatment on Tumor Volume

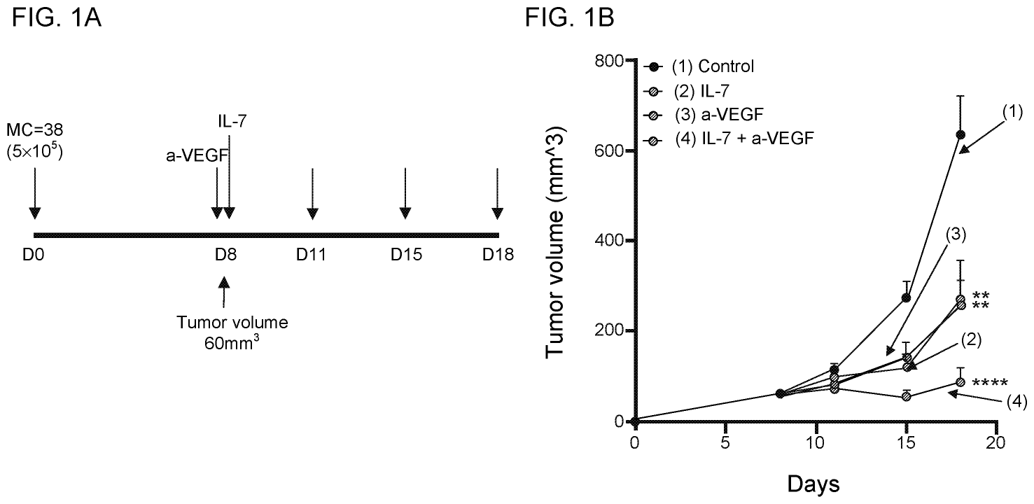

To assess the effect of IL-7 protein in combination with VEGF antagonist on tumor volume, a colon adenocarcinoma animal model was used. Briefly, MC-38 colon adenocarcinoma tumor cells (5×10⁵ cells, subcutaneously) were transplanted into each C57BL/6 mice. On day 8 post tumor inoculation (average tumor size of about 60 mm³), the animals were treated with the IL-7-HyFc protein and an antagonistic anti-VEGF antibody (aflibercept), alone or in combination. Control animals were treated with IL-7 formulating buffer and isotype control antibody. The IL-7 protein was administered one time (i.e., day 8 post tumor inoculation) (intramuscularly; 10 mg/kg per mouse). The antagonistic anti-VEGF antibody was administered twice a week for two weeks (i.e., days 8, 11, 15, and 18 post tumor inoculation) (intravenously; 10 mg/kg per mouse per dose). Then, tumor volume was monitored in the animals at various time points. FIG. 1A provides a graphical depiction of the dosing schedule and Table 2 (below) provides the different treatment groups.

TABLE 2
Treatment Groups

| Group | Treatment Regimen |
|---|---|
| “Group 1” = Control | IL-7-formulating buffer only |
| “Group 2” = IL-7 Protein alone | IL-7 protein + PBS |
| “Group 3” = Anti-VEGF alone | IL-7-formulating buffer + |
| | anti-VEGF antibody |
| “Group 4” = Combination | IL-7 protein + anti-VEGF antibody |

As shown in FIG. 1B, compared to the control animals (i.e., Group 1), animals treated with the IL-7 protein alone (i.e., Group 2) or the anti-VEGF antibody alone (i.e., Group 3) exhibited reduced tumor volume. However, in mice from the combination treatment group (i.e., Group 4), the reduction in tumor volume was much more dramatic. At day 18 post-tumor inoculation, the average tumor volume from the different treatment groups were as follows: Group 1: 635.30 mm³; Group 2: 269.46 mm³; Group 3: 257.99 mm³; and Group 4: 86.33 mm³.

The above results demonstrate that the combination treatment described herein (IL-7 protein in combination with a VEGF antagonist) could be useful in the treatment of certain cancers. Additionally, the data further shows that the IL-7 protein described herein is capable of improving the anti-tumor effects of VEGF antagonists, such as those that are commercially available and/or previously shown to have anti-tumor effects.

### Example 2: Analysis of the Anti-Tumor Effects of a Triple Combination Treatment of an IL-7 Protein, a VEGF Antagonist, and an Immune Checkpoint Inhibitor

To further assess the anti-tumor effects of the combination treatment described herein, a tumor animal model (e.g., colon adenocarcinoma animal model) will be treated with an IL-7 protein alone or in combination with a VEGF antagonist (e.g., anti-VEGF antibody). In some aspects, some of the animals will further receive an administration of an immune checkpoint inhibitor. Accordingly, in some aspects, some of the animals will be treated with a triple combination of an IL-7 protein, a VEGF antagonist, and an immune checkpoint inhibitor. After the administration, the anti-tumor immune response in the animals will be assessed, e.g., by monitoring tumor volume at various time points post treatment.

### Example 3: Effect of IL-7 Protein and VEGF Antagonist Combination Treatment in Human Glioblastoma Subjects

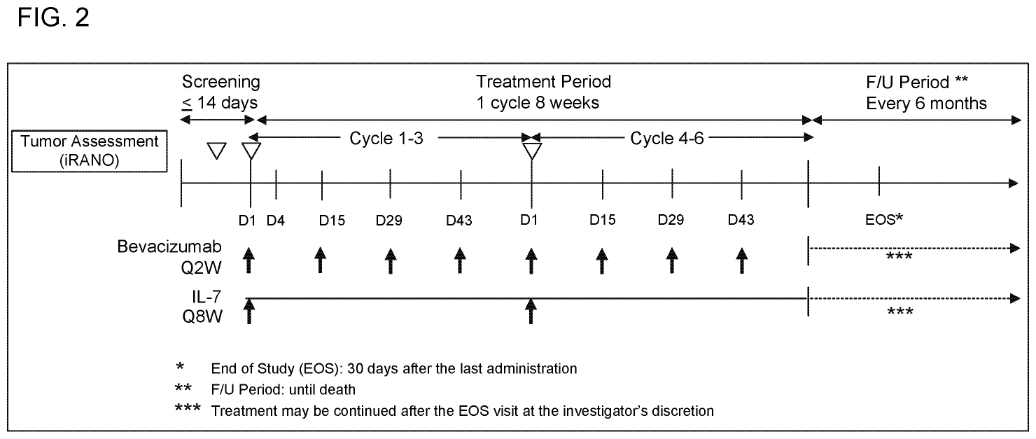

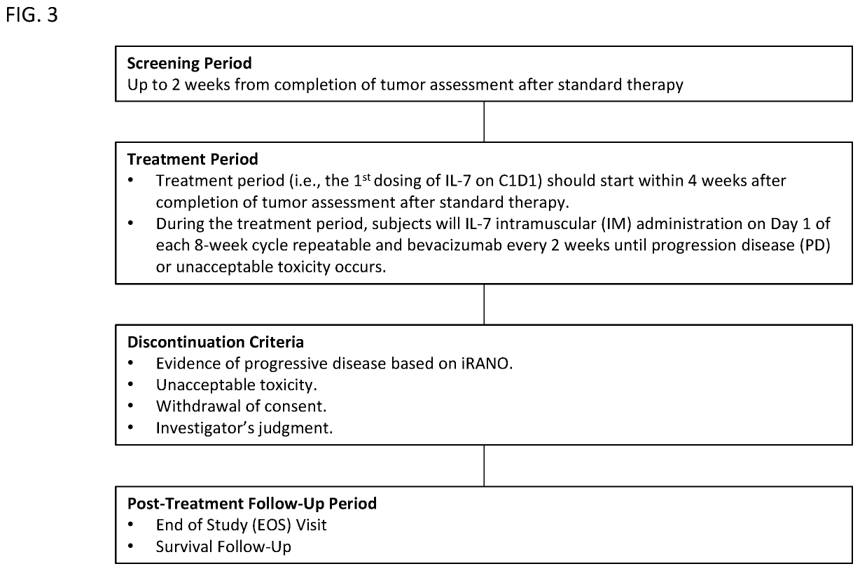

To assess both the efficacy and safety of the treatment regimens described herein, a phase 2 study is underway in human subjects suffering from recurrent glioblastoma. As shown in FIG. 2 and described further below, eligible subjects (see, e.g., inclusion and exclusion criteria described further below) have received an administration of an IL-7 protein described herein in combination with bevacizumab (i.e., VEGF antagonist) (also referred to herein as “study drug”). Eligible subjects are to receive up to six cycles (one cycle=eight weeks) of the combination treatment or until disease progression or development of unacceptable toxicity. Tumor evaluation are being conducted every eight weeks prior to the treatment administration. FIG. 3 provides an overall summary of the study.

### Test Subjects

Eligible subjects have been diagnosed with glioblastoma (e.g., recurrent glioblastoma) with confirmed disease progression after standard treatment (simultaneous chemotherapy and/or adjuvant chemotherapy with temozolomide).

### IL-7 Protein Administration

The IL-7 protein is administered to eligible subjects via intramuscular administration at a dose of 1,200 μg/kg and at a dosing interval of 8 weeks. As noted above, subjects without serious toxicity and without clinically clear evidence of disease progression are to receive up to 6 cycles of the IL-7 protein (based on the investigator's clinical significance and risk assessment). At each dose step, the amount of the IL-7 protein administered to a subject can be determined based on the subject's body weight on the first day of each cycle. There have been no restriction on the dosage of overweight subjects.

### Bevacizumab Administration

Bevacizumab is administered to eligible subjects via intravenous administration at a dose of 10 mg/kg every 2 weeks until disease progression or unacceptable toxicity occurs (as described elsewhere in the present Example).

### Dosage Adjustment

The dose of the IL-7 protein can be reduced to 960 μg/kg under the following conditions at the investigator's discretion: adverse skin reaction at injection site or systemically. If the subject experiences unacceptable toxicity (CTCAE grade 3 or higher) after IL-7 protein administration, the IL-7 protein administration is to be discontinued. If the toxicity subsides within 28 days, the IL-7 protein administration can be resumed. For subjects who experience clinically manageable toxicity (grade 3 immune-related adverse events) but exhibit signs of clear clinical benefit (e.g., tumor shrinkage or stable disease), IL-7 protein administration can be continued under close supervision. If after initial administration, absolute lymphocyte count (ALC) (related to the treatment) exceeds 20,000 cells/mm³ (corresponding to NCI CTC grade 3), further administration is to be temporarily discontinued. Administration can be resumed if the ALC decreases below the upper limit of normal level (4,000 cells/mm³).

As for the VEGF antagonist, the dosage can be adjusted when a subject's body weight changes more than about 10%. Otherwise, dosage adjustment is generally not recommended. In the event of an adverse event, administration of the VEGF agonist can be discontinued, temporarily or permanently, at the discretion of the investigator.

### Injection Site Reactions

Common adverse reactions that can occur at the site of injection include: allergic reactions (e.g., rash, hives, redness), itching, swelling, and erythema. Suitable doses of steroid (e.g., prednisolone at a dose of about 5-7.5 mg) can be used as a palliative treatment. Antihistamines can also be used for preventive purposes (e.g., administered to subjects at least 14 days before treatment).

### Prohibited Treatment

During the course of the present study, the following treatments are prohibited: (1) any concomitant treatment, including chemotherapy, hormone therapy, immunotherapy, radiotherapy, investigational drug or herbal therapy, approved or experimental for the treatment of cancer; (2) immunosuppressants, including but not limited to cyclophosphamide, azathioprine, methotrexate. and thalidomide; (3) granulocyte colony-stimulating factor and granulocyte macrophage colony-stimulating factor (e.g., pegfilgrastim); and (4) immune stimulants (e.g., IFN-α, IFN-γ, and IL-2).

### Inclusion Criteria

Inclusion criteria for determining a subject's eligibility included the following: (1) age≥19 years; (2) histologically diagnosed glioblastoma patients who have been confirmed the progression of disease after attempting standard therapy (RT/CCRT and/or adjuvant chemotherapy (TMZ)); (3) Karnofsky Performance Status; KPS≥60 or ECOG status 0-2; (4) life expectancy of greater than 12 weeks; and (5) adequate hematologic and end organ function.

### Exclusion Criteria

Subjects who exhibit one or more of the following have been excluded for the study: (1) malignancies other than disease under study within 5 years prior to the first dose of study drug; (2) received bevacizumab or other VEGF antagonists prior to the current study; (3) body mass index (BMI)≥30 kg/m²; (4) confirmed intracranial hemorrhage with non-contrast CT or MRI; (5) clinically significant cardiovascular disease; (6) history of arterial or venous thromboembolism 6 months prior to the current study; (7) uncontrolled hypertension (blood pressure≥150/90 mmHg with appropriate antihypertensive therapy); (8) history of hypertensive crisis or hypertensive encephalopathy; (9) receiving therapeutic anticoagulation (except low molecular weight heparin or warfarin); (10) pregnancy or breastfeeding; (11) active virus infection; (12) autoimmune disease/syndrome; (13) administration of a live, attenuated vaccine within 4 weeks before Cycle 1, Day 1 or anticipation that such a live attenuated vaccine will be required during the current study; (14) history of severe allergic, anaphylactic, or other hypersensitivity reactions to chimeric or humanized antibodies or fusion proteins; (15) severe infection during the screening period, including but not limited to complications of infection, bacteremia or severe pneumonia; and (16) prior allogeneic bone marrow transplantation or prior solid organ transplantation.

### Example 4: Interim Results of Phase 2 Study Involving IL-7 Protein and VEGF Antagonist Combination Treatment in Human Glioblastoma Subjects

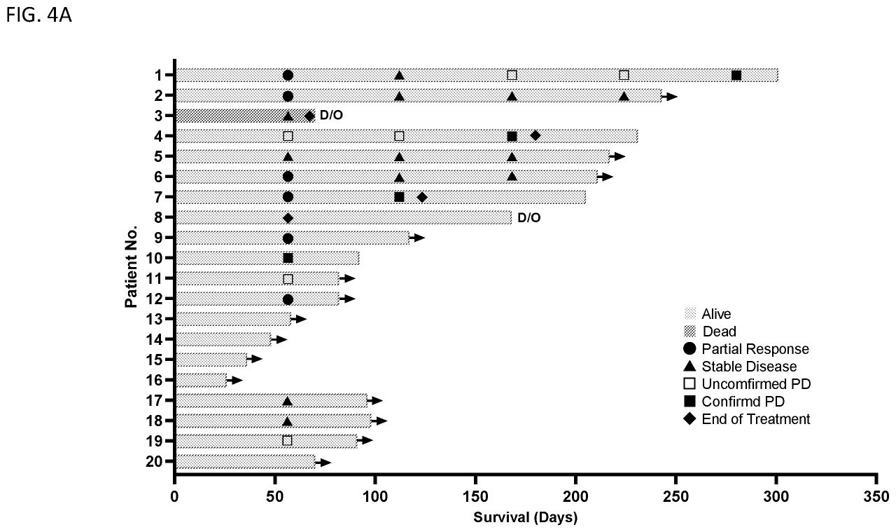

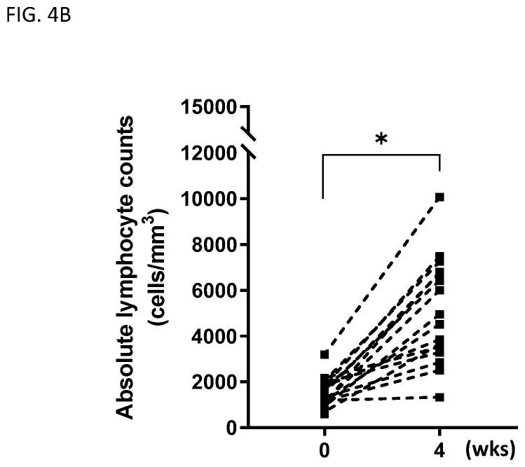

Further to Example 3 provided above, FIGS. 4A and 4B provide interim results of the study. There were 20 subjects enrolled in the study, and as shown in FIG. 4A, 14 subjects are still receiving treatment (patient nos. 2, 5, 6, 9, and 11-20) and four subjects have confirmed progression (patient nos. 1, 4, 7, and 10). Of those with confirmed progression, two subjects have been withdrawn from the study (patient nos. 4 and 7). And, as shown in FIG. 4B, at 4 weeks post initial administration, there was a significant increase in absolute lymphocyte count in nearly all subjects. Additional results (e.g., clinical response and survival status) are underway.

The above results show the therapeutic potential of the combination treatment described herein in treating cancers such as glioblastoma.

What is claimed is:

1. A method of treating a tumor in a subject in need thereof, comprising administering to the subject an IL-7 protein in combination with an antagonist of a vascular endothelial growth factor (“VEGF”) (“VEGF antagonist”).

2. The method of claim 1, wherein a tumor volume is reduced in the subject after the administration.

3. The method of claim 2, wherein the tumor volume is reduced by at least about 5%, at least about 10%, at least about 20%, at least about 30%, at least about 40%, at least about 50%, at least about 60%, at least about 70%, at least about 80%, at least about 90%, or about 100% after the administration.

4. A method of increasing an anti-tumor immune response in a subject in need thereof, comprising administering to the subject an IL-7 protein in combination with an antagonist of a vascular endothelial growth factor (“VEGF”) (“VEGF antagonist”).

5. The method of claim 4, wherein the anti-tumor immune response in the subject is increased by at least about 1-fold, at least about 2-fold, at least about 3-fold, at least about 4-fold, at least about 5-fold, at least about 10-fold, at least about 15-fold, at least about 20-fold, at least about 25-fold, at least about 30-fold, at least about 40-fold, or at least about 50-fold after the administration.

6. The method of claim 4 or 5, wherein an increased anti-tumor immune response comprises: (i) a decrease in a tumor volume; (ii) an increase in an effector activity of a tumor-specific T cell; (iii) an increase in the number of tumor-specific effector T cells; (iv) an increase in the number of tumor-infiltrating lymphocytes (TILs) in a tumor; (v) a decrease in the number of myeloid-derived suppressor cells (MDSCs) in a tumor; (vi) a decrease in the number of regulatory T cells (Tregs) in a tumor; (vii) an increase in duration of survival of the subject; (viii) a decrease in expression of TOX on CD8+ T cells in a tumor, tumor-draining lymph node (TDLN), or both; (ix) an increase in the number of stem-like T cells in a tumor, TDLN, or both; or (x) a combination thereof.

7. The method of any one of claims 1 to 6, wherein the IL-7 protein and the VEGF antagonist are administered to the subject concurrently.

8. The method of any one of claims 1 to 6, wherein the IL-7 protein and the VEGF antagonist are administered to the subject sequentially.

9. The method of any one of claims 1 to 8, wherein an additional therapeutic agent is administered to the subject.

10. The method of claim 9, wherein the additional therapeutic agent comprises an immune checkpoint inhibitor, an immune checkpoint activator, a standard care of treatment, or a combination thereof.

11. The method of claim 10, wherein the immune checkpoint inhibitor comprises a CTLA-4 antagonist (e.g., anti-CTLA-4 antibody), PD-1 antagonist (e.g., anti-PD-1 antibody, anti-PD-L1 antibody), TIM-3 antagonist (e.g., anti-TIM-3 antibody), or a combination thereof.

12. The method of claim 10 or 11, wherein the immune checkpoint activator comprises an OX40 agonist (e.g., anti-OX40 antibody), LAG-3 agonist (e.g. anti-LAG-3 antibody), 4-1BB (CD137) agonist (e.g., anti-CD137 antibody), GITR agonist (e.g., anti-GITR antibody), or a combination thereof.

13. The method of any one of claims 10 to 12, wherein the standard care of treatment comprises a chemotherapy, radiation, or both.

14. The method of any one of claims 1 to 13, wherein the IL-7 protein is not a wild type IL-7.

15. The method of any one of claims 1 to 14, wherein the IL-7 protein comprises an oligopeptide consisting of 1 to 10 amino acid residues.

16. The method of claim 15, wherein the oligopeptide comprises methionine (M), glycine (G), methionine-methionine (MM), glycine-glycine (GG), methionine-glycine (MG), glycine-methionine (GM), methionine-methionine-methionine (MMM), methionine-methionine-glycine (MMG), methionine-glycine-methionine (MGM), glycine-methionine-methionine (GMM), methionine-glycine-glycine (MGG), glycine-methionine-glycine (GMG), glycine-glycine-methionine (GGM), glycine-glycine-glycine (GGG), methionine-glycine-glycine-methionine (MGGM) (SEQ ID NO: 41), methionine-methionine-glycine-glycine (MMGG) (SEQ ID NO: 42), glycine-glycine-methionine-methionine (GGMM) (SEQ ID NO: 43), methionine-glycine-methionine-glycine (MGMG) (SEQ ID NO: 44), glycine-methionine-methionine-glycine (GMMG) (SEQ ID NO: 45), glycine-glycine-glycine-methionine (GGGM) (SEQ ID NO: 46), methionine-glycine-glycine-glycine (MGGG) (SEQ ID NO: 47), glycine-methionine-glycine-glycine (GMGG) (SEQ ID NO: 48), glycine-glycine-methionine-glycine (GGMG) (SEQ ID NO: 49), glycine-glycine-methionine-methionine-methionine (GGMMM) (SEQ ID NO: 50), glycine-glycine-glycine-methionine-methionine (GGGMM) (SEQ ID NO: 51), glycine-glycine-glycine-glycine-methionine (GGGGM) (SEQ ID NO: 52), methionine-glycine-methionine-methionine-methionine (MGMMM) (SEQ ID NO: 53), methionine-glycine-glycine-methionine-methionine (MGGMM) (SEQ ID NO: 54), methionine-glycine-glycine-glycine-methionine (MGGGM) (SEQ ID NO: 55), methionine-methionine-glycine-methionine-methionine (MMGMM) (SEQ ID NO: 56), methionine-methionine-glycine-glycine-methionine (MMGGM) (SEQ ID NO: 57), methionine-methionine-glycine-glycine-glycine (MMGGG) (SEQ ID NO: 58), methionine-methionine-methionine-glycine-methionine (MMMGM) (SEQ ID NO: 59), methionine-glycine-methionine-glycine-methionine (MGMGM) (SEQ ID NO: 60), glycine-methionine-glycine-methionine-glycine (GMGMG) (SEQ ID NO: 61), glycine-methionine-methionine-methionine-glycine (GMMMG) (SEQ ID NO: 62), glycine-glycine-methionine-glycine-methionine (GGMGM) (SEQ ID NO: 63), glycine-glycine-methionine-methionine-glycine (GGMMG) (SEQ ID NO: 64), glycine-methionine-methionine-glycine-methionine (GMMGM) (SEQ ID NO: 65), methionine-glycine-methionine-methionine-glycine (MGMMG) (SEQ ID NO: 66), glycine-methionine-glycine-glycine-methionine (GMGGM) (SEQ ID NO: 67), methionine-methionine-glycine-methionine-glycine (MMGMG) (SEQ ID NO: 68), glycine-methionine-methionine-glycine-glycine (GMMGG) (SEQ ID NO: 69), glycine-methionine-glycine-glycine-glycine (GMGGG) (SEQ ID NO: 70), glycine-glycine-methionine-glycine-glycine (GGMGG) (SEQ ID NO: 71), glycine-glycine-glycine-glycine-glycine (GGGGG) (SEQ ID NO: 72), or combinations thereof.

17. The method of claim 18, wherein the oligopeptide is methionine-glycine-methionine (MGM).

18. The method of any one of claims 1 to 17, wherein the IL-7 protein comprises a half-life extending moiety.

19. The method of claim 18, wherein the half-life extending moiety comprises an Fc, albumin, an albumin-binding polypeptide, Pro/Ala/Ser (PAS), a C-terminal peptide (CTP) of the R subunit of human chorionic gonadotropin, polyethylene glycol (PEG), long unstructured hydrophilic sequences of amino acids (XTEN), hydroxyethyl starch (HES), an albumin-binding small molecule, or a combination thereof.

20. The method of claim 19, wherein the half-life extending moiety is a Fc.

21. The method of claim 18 or 20, wherein the Fe is a hybrid Fe, comprising a hinge region, a CH2 domain, and a CH3 domain,
wherein the hinge region comprises a human IgD hinge region,
wherein the CH2 domain comprises a part of human IgD CH2 domain and a part of human IgG4 CH2 domain, and
wherein the CH3 domain comprises a part of human IgG4 CH3 domain.

22. The method of any one of claims 1 to 21, wherein the IL-7 protein comprises an amino acid sequence having a sequence identity of at least about 70%, at least about 75%, at least about 80%, at least about 85%, at least about 90%, at least about 95%, at least about 96%, at least about 97%, at least about 98%, at least about 99%, or about 100% to the sequence set forth in any one of SEQ ID NOs: 1-6, 15-26, and 80-85.

23. The method of any one of claims 1 to 22, wherein the VEGF antagonist comprises an antibody, or an antigen-binding fragment thereof, that specifically binds to the VEGF (“anti-VEGF antibody”), a polynucleotide encoding the anti-VEGF antibody, an antisense oligonucleotide, a siRNA, a shRNA, a miRNA, a dsRNA targeting VEGF, an aptamer, a PNA, or a vector comprising thereof.

24. The method of claim 23, wherein the VEGF antagonist is an anti-VEGF antibody.

25. The method of claim 24, wherein the anti-VEGF antibody comprises aflibercept (EYLEA® and ZALTRAP®), bevacizumab (AVASTIN®, ZIRABEV®, and MVASI®), ranibizumab (LUCENTIS®, BYOOVIZ®, SUSVIMO®), or a combination thereof.

26. The method of any one of claims 1 to 25, wherein the tumor is derived from a cancer comprising a breast cancer, head and neck cancer, uterine cancer, brain cancer, skin cancer, renal cancer, lung cancer, colorectal cancer, prostate cancer, liver cancer, bladder cancer, kidney cancer, pancreatic cancer, thyroid cancer, esophageal cancer, eye cancer, stomach (gastric) cancer, gastrointestinal cancer, ovarian cancer, carcinoma, sarcoma, leukemia, lymphoma, myeloma, or a combination thereof.

27. The method of claim 26, wherein the brain cancer comprises a glioblastoma.

28. The method of claim 27, wherein the glioblastoma comprise a recurrent glioblastoma.

29. The method of claim 27 or 28, wherein the glioblastoma is refractory to a standard of care therapy.

30. The method of any one of claims 26 to 29, wherein the skin cancer comprises a Merkel cell carcinoma (MCC), basal cell carcinoma (BCC), cutaneous squamous cell carcinoma (cSCC), melanoma, or a combination thereof.

31. The method of any one of claims 1 to 30, wherein the IL-7 protein is administered to the subject intramuscularly, parenthetically, subcutaneously, ophthalmic, intravenously, intraperitoneally, intradermally, intraorbitally, intracerebrally, intracranially, intraspinally, intraventricular, intrathecally, intracistemally, intracapsularly, or intratumorally.

32. The method of any one of claims 1 to 31, wherein the VEGF antagonist is administered to the subject intravenously, parenthetically, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally.

33. The method of any one of claims 9 to 32, wherein the additional therapeutic agent is administered to the subject parenthetically, intravenously, intramuscularly, subcutaneously, ophthalmic, intraperitoneally, intradermally, intraorbitally, intracerebrally, intracranially, intraspinally, intraventricular, intrathecally, intracistemally, intracapsularly, or intratumorally.

34. The method of any one of claims 1 to 33, wherein the IL-7 protein is administered at a dose of greater than about 600 μg/kg, greater than about 700 μg/kg, greater than about 800 μg/kg, greater than about 900 μg/kg, greater than about 1,000 μg/kg, greater than about 1,100 μg/kg, greater than about 1,200 μg/kg, greater than about 1,300 μg/kg, greater than about 1,400 μg/kg, greater than about 1,500 μg/kg, greater than about 1,600 μg/kg, greater than about 1,700 μg/kg, greater than about 1,800 μg/kg, greater than about 1,900 μg/kg, or greater than about 2,000 μg/kg.

35. The method of any one of claims 1 to 34, wherein the IL-7 protein is administered at a dose of between about 610 μg/kg and about 1,200 μg/kg, between about 650 μg/kg and about 1,200 μg/kg, between about 700 μg/kg and about 1,200 μg/kg, between about 750 μg/kg and about 1,200 μg/kg, between about 800 μg/kg and about 1,200 μg/kg, between about 850 μg/kg and about 1,200 μg/kg, between about 900 μg/kg and about 1,200 μg/kg, between about 950 μg/kg and about 1,200 μg/kg, between about 1,000 μg/kg and about 1,200 μg/kg, between about 1,050 μg/kg and about 1,200 μg/kg, between about 1,100 μg/kg and about 1,200 μg/kg, between about 1,200 μg/kg and about 2,000 μg/kg, between about 1,300 μg/kg and about 2,000 μg/kg, between about 1,500 μg/kg and about 2,000 μg/kg, between about 1,700 μg/kg and about 2,000 μg/kg, between about 610 μg/kg and about 1,000 μg/kg, between about 650 μg/kg and about 1,000 μg/kg, between about 700 μg/kg and about 1,000 μg/kg, between about 750 μg/kg and about 1,000 μg/kg, between about 800 μg/kg and about 1,000 μg/kg, between about 850 μg/kg and about 1,000 μg/kg, between about 900 μg/kg and about 1,000 μg/kg, or between about 950 μg/kg and about 1,000 μg/kg.

36. The method of any one of claims 1 to 35, wherein the IL-7 protein is administered at a dose of between about 700 μg/kg and about 900 μg/kg, between about 750 μg/kg and about 950 μg/kg, between about 700 μg/kg and about 850 μg/kg, between about 750 μg/kg and about 850 μg/kg, between about 700 μg/kg and about 800 μg/kg, between about 800 μg/kg and about 900 μg/kg, between about 750 μg/kg and about 850 μg/kg, or between about 850 μg/kg and about 950 μg/kg.

37. The method of any one of claims 1 to 36, wherein the IL-7 protein is administered at a dose of about 650 μg/kg, about 680 μg/kg, about 700 μg/kg, about 720 μg/kg, about 740 μg/kg, about 750 μg/kg, about 760 μg/kg, about 780 μg/kg, about 800 μg/kg, about 820 μg/kg, about 840 μg/kg, about 850 μg/kg, about 860 μg/kg, about 880 μg/kg, about 900 μg/kg, about 920 μg/kg, about 940 μg/kg, about 950 μg/kg, about 960 μg/kg, about 980 μg/kg, about 1,000 μg/kg, about 1,020 μg/kg, about 1,040 μg/kg, about 1,060 μg/kg, about 1,080 μg/kg, about 1,100 μg/kg, about 1,120 μg/kg, about 1,140 μg/kg, about 1,160 μg/kg, about 1,180 μg/kg, about 1,200 μg/kg, about 1,220 μg/kg, about 1,240 μg/kg, about 1,260 μg/kg, about 1,280 μg/kg, about 1,300 μg/kg, about 1,320 μg/kg, about 1,340 μg/kg, about 1,360 μg/kg, about 1,380 μg/kg, about 1,400 μg/kg, about 1,420 μg/kg, about 1,440 μg/kg, about 1,460 μg/kg, about 1,480 μg/kg, about 1,500 μg/kg, about 1,520 μg/kg, about 1,540 μg/kg, about 1,560 μg/kg, about 1,580 μg/kg, about 1,600 μg/kg, about 1,620 μg/kg, about 1,640 μg/kg, about 1,660 μg/kg, about 1,680 μg/kg, about 1,700 μg/kg, about 1,720 μg/kg, about 1,740 μg/kg, about 1,760 μg/kg, about 1,780 μg/kg, about 1,800 μg/kg, about 1,820 μg/kg, about 1,840 μg/kg, about 1,860 μg/kg, about 1,880 μg/kg, about 1,900 μg/kg, about 1,920 μg/kg, about 1,940 μg/kg, about 1,960 μg/kg, about 1,980 μg/kg, or about 2,000 μg/kg.

38. The method of any one of claims 1 to 37, wherein the TL-7 protein is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

39. The method of claim 38, wherein the IL-7 protein is administered at a dosing frequency of once in eight weeks.

40. The method of any one of claims 1 to 39, wherein the VEGF antagonist is administered to the subject at a dose of about 0.1 mg/kg to about 20 mg/kg.

41. The method of any one of claims 1 to 40, wherein the VEGF antagonist is administered at a dosing frequency of once a week, once in two weeks, once in three weeks, once in four weeks, once in five weeks, once in six weeks, once in seven weeks, once in eight weeks, once in nine weeks, once in 10 weeks, once in 11 weeks, or once in 12 weeks.

42. The method of claim 41, wherein the VEGF antagonist is administered at a dosing frequency of once in two weeks.

43. The method of any one of claims 25 to 42, wherein the anti-VEGF antibody is bevacizumab.

44. The method of claim 43, wherein the bevacizumab is administered at a dose of about 5 mg/kg to about 15 mg/kg.

45. The method of claim 44, wherein the bevacizumab is administered at a dose of about 5 mg/kg, about 7.5 mg/kg, about 10 mg/kg, about 12.5 mg/kg, or about 15 mg/kg.

46. The method of any one of claims 25 to 42, wherein the anti-VEGF antibody is aflibercept.

47. The method of claim 46, wherein the aflibercept is administered at a dose of about 4 mg/kg to about 15 mg/kg.

48. The method of claim 47, wherein the aflibercept is administered at a dose of about 4 mg/kg.


SEQUENCE LISTING (95 sequences)

SEQ ID NO: 1 - protein, 177 aa, Homo sapiens
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, Rattus norvegicus
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, Mus musculus
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, Chlorocebus aethiops
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, Bos taurus
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, Ovis aries
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, synthetic construct
  source 1..384: note = Human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, synthetic construct
  source 1..327: note = Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, synthetic construct
  source 1..245: note = hyFc
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, synthetic construct
  source 1..245: note = hyFcM1
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, synthetic construct
  source 1..245: note = hyFcM2
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, synthetic construct
  source 1..245: note = hyFcM3
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, synthetic construct
  source 1..245: note = hyFcM4
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, synthetic construct
  source 1..243: note = mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, synthetic construct
  source 1..153: note = modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, synthetic construct
  source 1..154: note = modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, synthetic construct
  source 1..155: note = modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, synthetic construct
  source 1..155: note = modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, synthetic construct
  source 1..155: note = modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, synthetic construct
  source 1..156: note = modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, synthetic construct
  source 1..398: note = modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, synthetic construct
  source 1..399: note = modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, synthetic construct
  source 1..400: note = modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, synthetic construct
  source 1..400: note = modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, synthetic construct
  source 1..401: note = modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, synthetic construct
  source 1..397: note = human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, synthetic construct
  source 1..395: note = human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, synthetic construct
  source 1..531: mol_type = other DNA; note = human IL-7
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, synthetic construct
  source 1..534: mol_type = other DNA; note = modified IL-7(M)
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, synthetic construct
  source 1..537: mol_type = other DNA; note = modified IL-7(MM)
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, synthetic construct
  source 1..540: mol_type = other DNA; note = modified IL-7(MMM)
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, synthetic construct
  source 1..540: mol_type = other DNA; note = modified IL-7(MGM)
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, synthetic construct
  source 1..540: mol_type = other DNA; note = modified IL-7(DDD)
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, synthetic construct
  source 1..543: mol_type = other DNA; note = modified IL-7(MMMM)
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, synthetic construct
  source 1..1284: mol_type = other DNA; note = modified IL-7(M) fused hyFc
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, synthetic construct
  source 1..1272: mol_type = other DNA; note = modified IL-7(MM) fused hyFc
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, synthetic construct
  source 1..1275: mol_type = other DNA; note = modified IL-7(MMM) fused hyFc
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, synthetic construct
  source 1..1275: mol_type = other DNA; note = modified IL-7(MGM) fused hyFc
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, synthetic construct
  source 1..1278: mol_type = other DNA; note = modified IL-7(MMMM) fused hyFc
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, synthetic construct
  source 1..4: note = synthetic oligopeptide fragment
MMMM

SEQ ID NO: 41 - protein, 4 aa, synthetic construct
  source 1..4: note = synthetic oligopeptide fragment
MGGM

SEQ ID NO: 42 - protein, 4 aa, synthetic construct
  source 1..4: note = synthetic oligopeptide fragment
MMGG

SEQ ID NO: 43 - protein, 4 aa, synthetic construct
  source 1..4: note = synthetic oligopeptide fragment
GGMM

SEQ ID NO: 44 - protein, 4 aa, synthetic construct
  source 1..4: note = synthetic oligopeptide fragment
MGMG

SEQ ID NO: 45 - protein, 4 aa, synthetic construct
  source 1..4: note = synthetic oligopeptide fragment
GMMG

SEQ ID NO: 46 - protein, 4 aa, synthetic construct
  source 1..4: note = synthetic oligopeptide fragment
GGGM

SEQ ID NO: 47 - protein, 4 aa, synthetic construct
  source 1..4: note = synthetic oligopeptide fragment
MGGG

SEQ ID NO: 48 - protein, 4 aa, synthetic construct
  source 1..4: note = synthetic oligopeptide fragment
GMGG

SEQ ID NO: 49 - protein, 4 aa, synthetic construct
  source 1..4: note = synthetic oligopeptide fragment
GGMG

SEQ ID NO: 50 - protein, 5 aa, synthetic construct
  source 1..5: note = synthetic oligopeptide fragment
GGMMM

SEQ ID NO: 51 - protein, 5 aa, synthetic construct
  source 1..5: note = synthetic oligopeptide fragment
GGGMM

SEQ ID NO: 52 - protein, 5 aa, synthetic construct
  source 1..5: note = synthetic oligopeptide fragment
GGGGM

SEQ ID NO: 53 - protein, 5 aa, synthetic construct
  source 1..5: note = synthetic oligopeptide fragment
MGMMM

SEQ ID NO: 54 - protein, 5 aa, synthetic construct
  source 1..5: note = synthetic oligopeptide fragment
MGGMM

SEQ ID NO: 55 - protein, 5 aa, synthetic construct
  source 1..5: note = synthetic oligopeptide fragment
MGGGM

SEQ ID NO: 56 - protein, 5 aa, synthetic construct
  source 1..5: note = synthetic oligopeptide fragment
MMGMM

SEQ ID NO: 57 - protein, 5 aa, synthetic construct
  source 1..5: note = synthetic oligopeptide fragment
MMGGM

SEQ ID NO: 58 - protein, 5 aa, synthetic construct
  source 1..5: note = synthetic oligopeptide fragment
MMGGG

SEQ ID NO: 59 - protein, 5 aa, synthetic construct
  source 1..5: note = synthetic oligopeptide fragment
MMMGM

SEQ ID NO: 60 - protein, 5 aa, synthetic construct
  source 1..5: note = synthetic oligopeptide fragment
MGMGM

SEQ ID NO: 61 - protein, 5 aa, synthetic construct
  source 1..5: note = synthetic oligopeptide fragment
GMGMG

SEQ ID NO: 62 - protein, 5 aa, synthetic construct
  source 1..5: note = synthetic oligopeptide fragment
GMMMG

SEQ ID NO: 63 - protein, 5 aa, synthetic construct
  source 1..5: note = synthetic oligopeptide fragment
GGMGM

SEQ ID NO: 64 - protein, 5 aa, synthetic construct
  source 1..5: note = synthetic oligopeptide fragment
GGMMG

SEQ ID NO: 65 - protein, 5 aa, synthetic construct
  source 1..5: note = synthetic oligopeptide fragment
GMMGM

SEQ ID NO: 66 - protein, 5 aa, synthetic construct
  source 1..5: note = synthetic oligopeptide fragment
MGMMG

SEQ ID NO: 67 - protein, 5 aa, synthetic construct
  source 1..5: note = synthetic oligopeptide fragment
GMGGM

SEQ ID NO: 68 - protein, 5 aa, synthetic construct
  source 1..5: note = synthetic oligopeptide fragment
MMGMG

SEQ ID NO: 69 - protein, 5 aa, synthetic construct
  source 1..5: note = synthetic oligopeptide fragment
GMMGG

SEQ ID NO: 70 - protein, 5 aa, synthetic construct
  source 1..5: note = synthetic oligopeptide fragment
GMGGG

SEQ ID NO: 71 - protein, 5 aa, synthetic construct
  source 1..5: note = synthetic oligopeptide fragment
GGMGG

SEQ ID NO: 72 - protein, 5 aa, synthetic construct
  source 1..5: note = synthetic oligopeptide fragment
GGGGG

SEQ ID NO: 73 - protein, 232 aa, synthetic construct
  source 1..232: note = Isoform VEGF206 (UniProt: P15692-1)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 74 - protein, 215 aa, synthetic construct
  source 1..215: note = Isoform VEGF189 (UniProt: P15692-2)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 75 - protein, 209 aa, synthetic construct
  source 1..209: note = Isoform VEGF183 (UniProt: P15692-3)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 76 - protein, 191 aa, synthetic construct
  source 1..191: note = Isoform VEGF165 (UniProt: P15692-4)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 77 - protein, 174 aa, synthetic construct
  source 1..174: note = Isoform VEGF148 (UniProt: P15692-5)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 78 - protein, 171 aa, synthetic construct
  source 1..171: note = Isoform VEGF145 (UniProt: P15692-6)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVCDKPRR

SEQ ID NO: 79 - protein, 191 aa, synthetic construct
  source 1..191: note = Isoform VEGF165B (UniProt: P15692-8)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 80 - protein, 147 aa, synthetic construct
  source 1..147: note = Isoform VEGF121 (UniProt: P15692-9)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 81 - protein, 137 aa, synthetic construct
  source 1..137: note = Isoform VEGF111 (UniProt: P15692-10)
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 82 - protein, 371 aa, synthetic construct
  source 1..371: note = Isoform L-VEGF165 (UniProt: P15692-11)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 83 - protein, 327 aa, synthetic construct
  source 1..327: note = Isoform L-VEGF121 (UniProt: P15692-12)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKCDKPRR

SEQ ID NO: 84 - protein, 395 aa, synthetic construct
  source 1..395: note = Isoform L-VEGF189 (UniProt: P15692-13)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVPCGPCSERRKHLFVQDPQTCKCSCKNTDSRCKARQLELNERTCRCDKPRR

SEQ ID NO: 85 - protein, 412 aa, synthetic construct
  source 1..412: note = Isoform L-VEGF206 (UniProt: P15692-14)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 86 - protein, 371 aa, synthetic construct
  source 1..371: note = Isoform #15 (UniProt: P15692-15)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRSLTRKD

SEQ ID NO: 87 - protein, 389 aa, synthetic construct
  source 1..389: note = Isoform #16 (UniProt: P15692-16)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRPCGPCSERRKHLFVQDPQTCKCSCKNTDSRCKARQLELNERTCRCDKPRR

SEQ ID NO: 88 - protein, 354 aa, synthetic construct
  source 1..354: note = Isoform #17 (UniProt: P15692-17)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKM

SEQ ID NO: 89 - protein, 317 aa, synthetic construct
  source 1..317: note = Isoform #18 (UniProt: P15692-18)
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRCDKPRR

SEQ ID NO: 90 - protein, 152 aa, synthetic construct
  source 1..152: note = Human IL-7 Protein (without signal peptide)
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 91 - protein, 129 aa, synthetic construct
  source 1..129: note = Rat IL-7 Protein (without signal peptide)
DCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 92 - protein, 129 aa, synthetic construct
  source 1..129: note = Mouse IL-7 Protein (without signal peptide)
ECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 93 - protein, 152 aa, synthetic construct
  source 1..152: note = Monkey IL-7 Protein (without signal peptide)
DCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 94 - protein, 151 aa, synthetic construct
  source 1..151: note = Cow IL-7 Protein (without signal peptide)
DCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 95 - protein, 151 aa, synthetic construct
  source 1..151: note = Sheep IL-7 Protein (without signal peptide)
DCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH